SEQUENCE LISTING (32 sequences)

SEQ ID NO: 1 - DNA, 972 bp, Homo sapiens
atggggagccgcgtctcgcgggaagacttcgagtgggtctacaccgaccagccgcacgccgaccggcgccgggagatcctggcaaagtatccagagataaagtccttgatgaaacctgatcccaatttgatatggattataattatgatggttctcacccagttgggtgcattttacatagtaaaagacttggactggaaatgggtcatatttggggcctatgcgtttggcagttgcattaaccactcaatgactctggctattcatgagattgcccacaatgctgcctttggcaactgcaaagcaatgtggaatcgctggtttggaatgtttgctaatcttcctattgggattccatattcaatttcctttaagaggtatcacatggatcatcatcggtaccttggagctgatggcgtcgatgtagatattcctaccgattttgagggctggttcttctgtaccgctttcagaaagtttatatgggttattcttcagcctctcttttatgcctttcgacctctgttcatcaaccccaaaccaattacgtatctggaagttatcaataccgtggcacaggtcacttttgacattttaatttattactttttgggaattaaatccttagtctacatgttggcagcatctttacttggcctgggtttgcacccaatttctggacattttatagctgagcattacatgttcttaaagggtcatgaaacttactcatattatgggcctctgaatttacttaccttcaatgtgggttatcataatgaacatcatgatttccccaacattcctggaaaaagtcttccactggtgaggaaaatagcagctgaatactatgacaacctccctcactacaattcctggataaaagtactgtatgattttgtgatggatgatacaataagtccctactcaagaatgaagaggcaccaaaaaggagagatggtgctggagtaa

SEQ ID NO: 2 - protein, 323 aa, Homo sapiens
MGSRVSREDFEWVYTDQPHADRRREILAKYPEIKSLMKPDPNLIWIIIMMVLTQLGAFYIVKDLDWKWVIFGAYAFGSCINHSMTLAIHEIAHNAAFGNCKAMWNRWFGMFANLPIGIPYSISFKRYHMDHHRYLGADGVDVDIPTDFEGWFFCTAFRKFIWVILQPLFYAFRPLFINPKPITYLEVINTVAQVTFDILIYYFLGIKSLVYMLAASLLGLGLHPISGHFIAEHYMFLKGHETYSYYGPLNLLTFNVGYHNEHHDFPNIPGKSLPLVRKIAAEYYDNLPHYNSWIKVLYDFVMDDTISPYSRMKRHQKGEMVLE

SEQ ID NO: 3 - DNA, 1434 bp, Saccharomyces cerevisiae
atgtacaacagaaaagacagagatgttcacgagaggaaggaagatggtcaatctgagtttgaagcactgaacgggaccaacgcaattatgtccgataatagtaaagcgtattccataaagtttctgaccttcaatacatgggggttaaaatacgtctccaaacaccgtaaagaaagactcagagcaattgctgataaattggcgggccactcaatgcttacgccaatatctgacgagttgttgcccaatggtggagatagtaatgaaaacgaagattacgacgtgattgccttacaagaaatctggtgtgtggaagactggaagtatctagcttctgcgtgtgcctccaagtatccgtatcagcgtttgttccattctggtattctgacggggcctgggttggccatactgtccaaggtcccgatagagtcgacctttctttaccggttcccgataaacggtagaccgagtgcggtgttccgtggcgactggtacgtagggaaatctatagcaatcaccgtattgaacacaggaacccgccccattgcaataatgaacagtcacatgcacgccccatacgccaagcagggtgatgccgcctacttgtgccacagatcttgtcaggcctgggatttcagcaggctcattaagctttacaggcaggccggttatgcggtgattgtggtgggtgacttaaactccagaccgggctcactgccccacaaatttctcacgcaggaggccggcctggtcgactcctgggagcaattgcatgggaagcaagacttggcggtgatcgctcgtctgtctccattgcaacaattgcttaagggctgtaccacgtgcgattcgctgctcaacacatggagggcccaaagacaacccgatgaggcatgcaggttggattatgctcttatcgaccctgatttcttgcaaacagtagacgcaggtgtcaggttcactgaacggatccctcacctggactgcagtgtctctgaccattttgcatactcatgcacccttaacatcgtcccacagggcacagagtcccgtccatccacctccgttaagcgtgcgaagactcatgatagagagctgatcttgcagagatactccaactacgaaaccatgatagaatgcatccacacgtacttgaagacagcccaaagacagaaatttttccgtggcctacatttctgggcctcaatacttctcctaatagcgtcgttggtcgtgacaacgtttactgcaaacaaggcaggctggtcctccatcttctgggtccttttcgctattgctgtctccatctcgggcaccatcgacggtgccatctccttcttgtttggcaggtctgaaatcagagccctcatcgaagtcgaacaagaggttctggacgcggagcaccacctgcaaactttcttgagcgagaaatga

SEQ ID NO: 4 - protein, 477 aa, Saccharomyces cerevisiae
MYNRKDRDVHERKEDGQSEFEALNGTNAIMSDNSKAYSIKFLTFNTWGLKYVSKHRKERLRAIADKLAGHSMLTPISDELLPNGGDSNENEDYDVIALQEIWCVEDWKYLASACASKYPYQRLFHSGILTGPGLAILSKVPIESTFLYRFPINGRPSAVFRGDWYVGKSIAITVLNTGTRPIAIMNSHMHAPYAKQGDAAYLCHRSCQAWDFSRLIKLYRQAGYAVIVVGDLNSRPGSLPHKFLTQEAGLVDSWEQLHGKQDLAVIARLSPLQQLLKGCTTCDSLLNTWRAQRQPDEACRLDYALIDPDFLQTVDAGVRFTERIPHLDCSVSDHFAYSCTLNIVPQGTESRPSTSVKRAKTHDRELILQRYSNYETMIECIHTYLKTAQRQKFFRGLHFWASILLLIASLVVTTFTANKAGWSSIFWVLFAIAVSISGTIDGAISFLFGRSEIRALIEVEQEVLDAEHHLQTFLSEK

SEQ ID NO: 5 - DNA, 1050 bp, Saccharomyces cerevisiae
atgaacgtaacatcgaatgcaactgcagccggttcctttccactagcatttggtctcaagacctcatttgggtttatgcactatgccaaggcccctgccattaatttacgccccaaggaatccttgctgccggaaatgagtgatggtgtgctggccttggttgcgccggttgttgcctactgggcgttgtctggtatattccatgtaatagacactttccatctggctgagaagtacagaattcatccgagcgaagaggttgccaagaggaacaaggcgtcgagaatgcatgttttccttgaagtgattctacaacatatcatacagaccattgttggccttatctttatgcacttcgagccgatctacatgactgggtttgaagaaaatgccatgtggaagcttcgtgcagaccttcctcggattattccagatgccgctatttattacggctatatgtacggaatgtccgctttgaagatctttgcaggctttttattcgttgatacatggcaatactttttgcatagattgatgcatatgaataagaccttatacaaatggttccactctgttcatcatgaactatacgtgccatatgcttacggtgctcttttcaacaatcctgttgagggcttcttgttagatactttgggaaccggtattgccatgacgttaactcatttgactcacagagagcaaatcattctttttacctttgccaccatgaagactgtcgatgaccactgtgggtatgctttgccacttgacccattccaatggcttttccctaataacgctgtctatcacgatatccaccaccagcaatttggtatcaagacgaactttgctcaaccatttttcactttctgggacaatttgttccaaactaactttaaagggtttgaagaatatcaaaagaagcaaagacgtgtcaccatcgacaagtacaaagagtttttgcaagagagagaattggaaaagaaggagaaactcaaaaacttcaaagctatgaatgctgctgaaaatgaagtaaagaaagagaaataa

SEQ ID NO: 6 - protein, 349 aa, Saccharomyces cerevisiae
MNVTSNATAAGSFPLAFGLKTSFGFMHYAKAPAINLRPKESLLPEMSDGVLALVAPVVAYWALSGIFHVIDTFHLAEKYRIHPSEEVAKRNKASRMHVFLEVILQHIIQTIVGLIFMHFEPIYMTGFEENAMWKLRADLPRIIPDAAIYYGYMYGMSALKIFAGFLFVDTWQYFLHRLMHMNKTLYKWFHSVHHELYVPYAYGALFNNPVEGFLLDTLGTGIAMTLTHLTHREQIILFTFATMKTVDDHCGYALPLDPFQWLFPNNAVYHDIHHQQFGIKTNFAQPFFTFWDNLFQTNFKGFEEYQKKQRRVTIDKYKEFLQERELEKKEKLKNFKAMNAAENEVKKEK

SEQ ID NO: 7 - DNA, 1155 bp, Saccharomyces cerevisiae
atgtcgactaatacttccaagactttggaactgttttcaaaaaagacggtacaagaacacaatactgccaatgactgctgggtcacttatcaaaacagaaagatttatgacgtgaccaggtttttgagcgaacaccctggtggtgacgagtccatcttggactatgctggtaaggacattactgagatcatgaaagactcagatgtgcatgaacacagcgactccgcgtatgaaatccttgaggacgaatatttgattggttacttggcaactgacgaagaggcagcgagattgttgactaacaagaaccataaggttgaagtgcagttgtcagctgacggtactgagtttgactccactacttttgtaaaggagttgcccgccgaggagaaactaagtattgctacggactacagtaacgactacaaaaagcataaatttttggatctgaaccgtcctttgctgatgcagattctgcgtagtgatttcaagaaagatttttacgttgaccaaatccatagaccaagacattacggtaaggggtctgccccgctatttggtaatttcttggaaccattaactaaaacagcttggtgggttgttccagttgcttggttgcctgtagttgtgtaccacatgggtgttgctttgaagaacatgaaccagctatttgcatgtttcttgttctgtgtcggtgtctttgtttggactttgattgaatacggtcttcaccgtttcctatttcatttcgatgattggttacctgaaagtaacatcgcattcgccacacattttctactacatggttgccatcattacttgcccatggacaagtaccgtttagttatgccacctactctgttcgtcatcctttgtgctccattttacaagttggtatttgctctgctgccactttattgggcttacgctggttttgctggcggtcttttcggttatgtctgttatgacgaatgtcatttcttcttgcaccactctaaattgcctcccttcatgcgtaagttgaaaaaatatcacctggaacatcattataaaaactaccaactgggatttggcgtcacatcctggttttgggacgaagtttttggcacctacttaggccccgatgccccattgtccaaaatgaaatatgaataa

SEQ ID NO: 8 - protein, 384 aa, Saccharomyces cerevisiae
MSTNTSKTLELFSKKTVQEHNTANDCWVTYQNRKIYDVTRFLSEHPGGDESILDYAGKDITEIMKDSDVHEHSDSAYEILEDEYLIGYLATDEEAARLLTNKNHKVEVQLSADGTEFDSTTFVKELPAEEKLSIATDYSNDYKKHKFLDLNRPLLMQILRSDFKKDFYVDQIHRPRHYGKGSAPLFGNFLEPLTKTAWWVVPVAWLPVVVYHMGVALKNMNQLFACFLFCVGVFVWTLIEYGLHRFLFHFDDWLPESNIAFATHFLLHGCHHYLPMDKYRLVMPPTLFVILCAPFYKLVFALLPLYWAYAGFAGGLFGYVCYDECHFFLHHSKLPPFMRKLKKYHLEHHYKNYQLGFGVTSWFWDEVFGTYLGPDAPLSKMKYE

SEQ ID NO: 9 - DNA, 954 bp, Saccharomyces cerevisiae
atgctgttcagctggccttatccagaagccccgattgaaggttattggggcaagccaacttctctgattgattggtgcgaggagaattatgtcgtatccccctatattgcagaatggtcaaatactattaccaatagtatattcttaatgaccgccttctattctacatatagcgcttggcgtaataagttagaaacaaggtatatattgataggaatggggttctcgctggttggtattggttcgtggttatttcatatgactttacagtatcgttatcaattgctagacgaactaccaatgctgtatgcgaccatcatcccatcgtggagtatttttgcagaaactcaagaaatcttgattaaggatgagaagaaaaggaaggaaagctcatttagaatccaaatggtcatttcttttatcatgtgtggtatagtcaccattttaacctggatttacgttgtcgtccaaaagccagcaattttccaagtcctttatggtatattgacgcttctagttgtggttctttctggctggctgacctactatcacgttcatgattcatttgcaaagaaaaatctttttattactatggttatgggcatgattccttttgtcattgggttcatttgctggcaactagatattcacctgtgttctttttggatctatatccggagaacatatttggccctgccattaggtgttctattggaactgcatgcttggtggcatcttttgaccggtactggtgtctatatcttcgtcgtgtatttgcaatatttgagaatattaacccatggaaatccaaatgacttcttatttatatggaggtggggatttttccctgagctggtaagaaagggcttaccgattggtacttcttattcactggagtatctggggccaattgtaaatacacaggtagatgatgaaacaaaaaagaataactaa

SEQ ID NO: 10 - protein, 317 aa, Saccharomyces cerevisiae
MLFSWPYPEAPIEGYWGKPTSLIDWCEENYVVSPYIAEWSNTITNSIFLMTAFYSTYSAWRNKLETRYILIGMGFSLVGIGSWLFHMTLQYRYQLLDELPMLYATIIPSWSIFAETQEILIKDEKKRKESSFRIQMVISFIMCGIVTILTWIYVVVQKPAIFQVLYGILTLLVVVLSGWLTYYHVHDSFAKKNLFITMVMGMIPFVIGFICWQLDIHLCSFWIYIRRTYLALPLGVLLELHAWWHLLTGTGVYIFVVYLQYLRILTHGNPNDFLFIWRWGFFPELVRKGLPIGTSYSLEYLGPIVNTQVDDETKKNN

SEQ ID NO: 11 - DNA, 44 bp, Artificial Sequence
  Description of Artificial Sequence Synthetic primer
ccttctctagaggatccatggggagccgcgtctcgcgggaagac

SEQ ID NO: 12 - DNA, 45 bp, Artificial Sequence
  Description of Artificial Sequence Synthetic primer
ccttcgaattccccgggccaggggagcttctgagcatcactggtc

SEQ ID NO: 13 - DNA, 25 bp, Artificial Sequence
  Description of Artificial Sequence Synthetic primer
atgtacaacagaaaagacagagatg

SEQ ID NO: 14 - DNA, 30 bp, Artificial Sequence
  Description of Artificial Sequence Synthetic primer
aaggtacctcatttctcgctcaagaaagtt

SEQ ID NO: 15 - DNA, 66 bp, Artificial Sequence
  Description of Artificial Sequence Synthetic primer
ctccggcttctgcggtttttcttagtctttccgcaccaattttcacaggaattcccggggatccgg

SEQ ID NO: 16 - DNA, 68 bp, Artificial Sequence
  Description of Artificial Sequence Synthetic primer
ggataataaatacaaacgtgggaagtcggagacattgcctttacccagcaagctagcttggctgcagg

SEQ ID NO: 17 - DNA, 31 bp, Artificial Sequence
  Description of Artificial Sequence Synthetic primer
ctccggcttctgcggtttttcttagtctttc

SEQ ID NO: 18 - DNA, 28 bp, Artificial Sequence
  Description of Artificial Sequence Synthetic primer
ggaagtcggagacattgcctttacccag

SEQ ID NO: 19 - DNA, 27 bp, Artificial Sequence
  Description of Artificial Sequence Synthetic primer
cgaattcagccgaaaacagtcttgctt

SEQ ID NO: 20 - DNA, 36 bp, Artificial Sequence
  Description of Artificial Sequence Synthetic primer
acgaggctgggatccgcttaccaccgcttttagtgc

SEQ ID NO: 21 - DNA, 36 bp, Artificial Sequence
  Description of Artificial Sequence Synthetic primer
ggtggtaagcggatcccagcctcgtccaaaattgtc

SEQ ID NO: 22 - DNA, 27 bp, Artificial Sequence
  Description of Artificial Sequence Synthetic primer
cgaattcttgccaacctgatctgtgaa

SEQ ID NO: 23 - DNA, 26 bp, Artificial Sequence
  Description of Artificial Sequence Synthetic primer
cgaattccccagaggcaaagatgtta

SEQ ID NO: 24 - DNA, 39 bp, Artificial Sequence
  Description of Artificial Sequence Synthetic primer
tggatggcacggatccgaaaggcacacctgtcattatgg

SEQ ID NO: 25 - DNA, 34 bp, Artificial Sequence
  Description of Artificial Sequence Synthetic primer
tgtgcctttcggatccgtgccatccatttgaatc

SEQ ID NO: 26 - DNA, 30 bp, Artificial Sequence
  Description of Artificial Sequence Synthetic primer
cgaattccttttatgatgggagtaactgct

SEQ ID NO: 27 - DNA, 30 bp, Artificial Sequence
  Description of Artificial Sequence Synthetic primer
ctccatgtcgcttaccaccgcttttagtgc

SEQ ID NO: 28 - DNA, 31 bp, Artificial Sequence
  Description of Artificial Sequence Synthetic primer
cgctatactgcagcctcgtccaaaattgtca

SEQ ID NO: 29 - DNA, 30 bp, Artificial Sequence
  Description of Artificial Sequence Synthetic primer
gtggtaagcgacatggaggcccagaatacc

SEQ ID NO: 30 - DNA, 31 bp, Artificial Sequence
  Description of Artificial Sequence Synthetic primer
gacgaggctgcagtatagcgaccagcattca

SEQ ID NO: 31 - DNA, 20 bp, Artificial Sequence
  Description of Artificial Sequence Synthetic primer
tccagcaccatctctccttt

SEQ ID NO: 32 - DNA, 20 bp, Artificial Sequence
  Description of Artificial Sequence Synthetic primer
agtgggtctacaccgaccag

(19) United States
(12) Patent Application Publication
(10) Pub. No.: US 2009/0325247 A1
(43) Pub. Date: Dec. 31, 2009

(54) METHODS FOR PRODUCING CERAMIDE USING TRANSFORMED YEAST

(75) Inventors: Yukiko Kodama, Osaka (JP); Hideaki Nagano, Osaka (JP); Koichi Funato, Hiroshima (JP)

Correspondence Address: DRINKER BIDDLE & REATH (DC), 1500 K STREET, N.W., SUITE 1100, WASHINGTON, DC, 20005-1209, US

(73) Assignees: Suntory Limited, Osaka-shi (JP); Hiroshima University, Higashi-Hiroshima-shi (JP)

(21) Appl. No.: 12/096,153

(22) PCT Filed: Dec. 1, 2006
(86) PCT No.: PCT/JP2006/324080

§ 371 Date: Jun. 4, 2008

(30) Foreign Application Priority Data
Dec. 5, 2005 (JP) 2005-351366

(51) Int. Cl. C12P 7/64

(52) U.S. Cl. 435/134

(57) ABSTRACT

The present invention provides methods for producing human ceramide in a yeast cell.

The methods of the present invention comprise:
1) introducing the sphingoid Δ4-desaturase gene (DES1) by transformation of the yeast cell; and
2) abolishing the expression of the yeast sphinganine C4-hydroxylase gene (SUR2) by transformation of the yeast cell.

### TECHNICAL FIELD

The present application claims priority based on Japanese Patent Application No. 2005-351366 filed on Dec. 5, 2005.

The present invention relates to methods for producing human ceramide in yeast cells.

### BACKGROUND ART

A tissue known as stratum corneum exists on the outermost layer of the skin, which has a moisturizing function for retaining moisture as well as a barrier function for protecting the skin against external stimulation. The stratum corneum consists of keratinocytes, natural moisturizing factors and intercellular lipids, among which ceramides account for approximately one-half of the total intercellular lipids and play a crucial role for these functions. For example, a common characteristic of atopic dermatitis and senile xerosis is a significant deterioration of moisturizing ability, which is known to mainly result from decreased ceramide levels due to lipid metabolic enzyme abnormalities. Ceramides have also been shown to enhance barrier function, provide whitening effect and inhibit melanogenesis. Ceramides can be externally supplied.

J Invest Dermatol. 96:523-526, 1991 (Non-patent document 1) and Arch Dermatol Res. 283:219-223, 1991 (Non-patent document 2) disclose “decreased ceramide levels in atopic dermatitis and senile xerosis”, J Dermatol Sci. 1:79-83, 1990 (Non-patent document 3) and Acta Derm Venereol. 74:337-340, 1994 (Non-patent document 4) disclose “decreased ceramide levels and lipid metabolic enzyme abnormalities”; Contact Dermatitis. 45:280-285, 2001 (Non-patent document 5) and J Eur Acad Dermatol Venereol. 16:587-594, 2002 (Non-patent document 6) disclose “restoration of barrier function by ceramides”; and Cell Signal 14:779-785, 2002 (Non-patent document 7) discloses “inhibition of melanogenesis by ceramides”.

Recently, ceramides have attracted great attention for use in medicines for skin diseases associated with dry sensitive skin or in cosmetics or health and/or cosmeceutical foods. In fact, a number of products such as cosmetics and food or supplements containing ceramides have already been commercialized, and the market for ceramide materials is continuing to grow.

Ceramide materials of animal origin such as cow were conventionally used, but are currently replaced by those of plant origin such as rice, wheat, soybean and potato because of problems of infections. A recent basic study (J. Clin. Invest. 112:1372-1382, 2003 (Non-patent document 8)) showed the importance of the structures of ceramides in the moisturizing and barrier functions of the skin, which raised questions about whether plant ceramides structurally different from human ceramides are highly functional lipids. Moreover, ceramides are present in animals and plants in minute amounts and are difficult to extract and purify, thus incurring low productivity and high cost, and therefore, it is highly desirable to develop a new production technique capable of overcoming these problems.

As shown in FIG. 1, it is known that dihydrosphingosine (DHS) biosynthesis and the subsequent reactions in the synthetic/metabolic pathway for sphingolipids widely differ between higher animal cells, including human cells and yeast cells. Each enzyme protein involved in various steps in the synthetic/metabolic pathway for sphingolipids and the gene encoding the protein have been known to some degree (Biochemistry. 41:15105-15114, 2002 (Non-patent document 9); J Biol Chem. 277:25512-25518, 2002 (Non-patent document 10); Yeast 9: 267-277, 1993 (Non-patent document 11); J Biol Chem 272:29704-29710, 1997 (Non-patent document 12); J Biol Chem 275:31369-31378, 2000 (Non-patent document 13); J Biol Chem 275:39793-39798, 2000 (Non-patent document 14)).

### REFERENCES

Non-patent document 1: J Invest Dermatol. 96:523-526, 1991.
Non-patent document 2: Arch Dermatol Res. 283:219-223, 1991.
Non-patent document 3: J Dermatol Sci. 1:79-83, 1990.
Non-patent document 4: Acta Derm Venereol. 74:337-340, 1994.
Non-patent document 5: Contact Dermatitis. 45:280-285, 2001.
Non-patent document 6: J Eur Acad Dermatol Venereol. 16:587-594, 2002.
Non-patent document 7: Cell Signal 14:779-785, 2002.
Non-patent document 8: J. Clin. Invest. 112:1372-1382, 2003
Non-patent document 9; Biochemistry. 41:15105-15114, 2002.
Non-patent document 10: J Biol Chem. 277:25512-25518, 2002.
Non-patent document 11: Yeast 9: 267-277, 1993.
Non-patent document 12; J Biol Chem 272:29704-29710, 1997.
Non-patent document 13; J Biol Chem 275:31369-31378, 2000.
Non-patent document 14: J Biol Chem 275:39793-39798, 2000.

### DISCLOSURE OF THE INVENTION

### Problems to be Solved by the Invention

An object of the present invention is to provide a method for producing human ceramide in a yeast cell. The method of the present invention comprises:

1) introducing the sphingoid Δ4-desaturase gene (DES1) by transformation of the yeast cell; and

2) abolishing the expression of the yeast sphinganine C4-hydroxylase gene (SUR2) by transformation of the yeast cell.

In the present invention, the sphingoid Δ4-desaturase gene (DES1) preferably has the amino acid sequence of SEQ ID NO: 2 or an amino acid sequence obtained by deletion, addition or substitution of one or more amino acid residues in SEQ ID NO: 2, and encodes a protein having sphingoid Δ4-desaturase activity.

In the present invention, the yeast sphinganine C4-hydroxylase gene (SUR2) preferably has the amino acid sequence of SEQ ID NO: 6 or an amino acid sequence obtained by deletion, addition or substitution of one or more amino acid residues In SEQ ID NO; 6, and encodes a protein having sphinganine C4-hydroxylase activity.

A preferred embodiment of the method of the present invention further comprises abolishing the expression of the yeast sphingolipid α-hydroxylase gene (SCS7) by transformation of the yeast cell.

A preferred embodiment of the method of the present invention further comprises abolishing the expression of the yeast alkaline dihydroceramidase gene (YDC1) by transformation of the yeast cell.

A preferred embodiment of the method of the present invention further comprises enhancing the expression of the yeast inositol phosphosphingolipid phospholipase C gene (ISC1) by transformation of the yeast cell.

### Means to Solve the Problems

As a result of careful studies to solve the problems described above, the present inventors have achieved the present invention.

The structures of ceramides depend on the types of enzymes possessed by cells, and vary between species. Yeasts possess no enzyme for synthesizing ceramide NS, which is a main ceramide in higher animals. Thus, necessary enzymes must be introduced into yeast cells to synthesize ceramide NS in yeast cells. Moreover, host cells have a host-derived ceramide synthetic pathway. Therefore, ceramide NS is selectively synthesized with high efficiency by inhibiting the host-derived ceramide synthetic pathway.

Specifically, in the synthetic/metabolic pathway for sphingolipids, reactions downstream of dihydrosphingosine (DHS) biosynthesis widely differ between higher animal cells including human cells and yeast cells, as shown in FIG. 1. More specifically, no human sphingoid base (sphingosine) having a double bond at C-4 of DHS is synthesized in budding yeast (genus Saccharomyces) due to the absence of the sphingoid Δ4-desaturase gene (DES1) (FIG. 2). Instead, phytosphingosine (PHS) is synthesized by hydroxylation of C-4 of DHS by an enzyme encoded by the sphinganine C4-hydroxylase gene (SUR2). Then, these sphingoid bases are converted into ceramides.

First, the present inventors thought it important to allow yeast cells to express a sphingoid Δ4-desaturase enzyme not present in yeast cells and to completely or even partially abolish sphinganine C4-hydroxylase enzyme activity in order to produce human ceramide in yeast cells. Thus, we initially 1) prepared the SUR2 gene disruption strain, and 2) introduced the human DES1 gene into the variant strain by transformation of yeast, thereby achieving the present invention. Thus, we first succeeded in producing human ceramide in yeast cells, which had not been achieved before the present invention.

We further optimized the above system to construct a system for efficiently producing human ceramide NS. Specifically, we succeeded in producing human ceramide more efficiently by transformation of yeast with any one of the following 3) to 5):

3) abolishing the expression of the yeast sphingolipid α-hydroxylase gene (SCS7) to prevent hydroxylation of ceramide NS;

4) abolishing the expression of the yeast alkaline dihydroceramidase gene (YDC1); or

5) enhancing the expression of the yeast inositol phosphosphingolipid phospholipase C gene (ISC1); or a combination of 3)-5).

Thus, the present invention provides methods for conveniently and efficiently producing human ceramide in yeast cells by including 1) and 2) above as essential features, and 3)-5) as additional features in preferred embodiments.

Accordingly, the present invention relates to methods for producing human ceramide in a yeast cell, comprising:

1) introducing the sphingoid Δ4-desaturase gene (DES1) by transformation of the yeast cell; and

2) abolishing the expression of the yeast sphinganine C4-hydroxylase gene (SUR2) by transformation of the yeast cell.

As used herein, human ceramide refers to ceramide NS having the structural formula shown as “target product” in FIG. 2, for example. In contrast, phytoceramide is yeast ceramide, which differs from the human ceramide in that the double bond at the 4-position of the human ceramide is substituted by a hydroxy group (FIG. 3).

Sphingoid Δ4-Desaturase Gene (DES1)

The methods of the present invention comprise introducing the sphingoid Δ4-desaturase gene (DES1) by transformation of the yeast cell, as essential feature 1).

DES1 preferably has, but is not limited to, the amino acid sequence of SEQ ID NO: 2 or an amino acid sequence obtained by deletion, addition or substitution of one or more amino acid residues in SEQ ID NO; 2, and encodes a protein having sphingoid Δ4-desaturase activity.

Genes (nucleic acids) that can be used in the present invention include genomic DNAs (including their corresponding cDNAs), chemically synthesized DNAs, DNAs amplified by PCR, and combinations thereof.

DES1 preferably has the nucleotide sequence of SEQ ID NO: 1. This is a nucleotide sequence encoding a human sphingoid Δ4-desaturase protein having the amino acid sequence of SEQ ID NO; 2, and it is disclosed in, for example, GenBank™: accession number AF466375.

One or more codons may encode the same amino acid, and this is called degeneracy of the genetic code. Thus, a DNA sequence not completely identical to SEQ ID NO: 1 may encode a protein having an amino acid sequence completely identical to SEQ ID NO: 2. Such a variant DNA sequence may result from silent mutation (e.g., occurring during PCR amplification), or can be the product of deliberate mutagenesis of a native sequence.

DES1 preferably encodes the amino acid sequence of SEQ ID NO: 2. However, it may also have an amino acid sequence obtained by deletion, addition or substitution of one or more amino acid residues. It is intended to encompass any homologous protein so long as it has sphingoid Δ4-desaturase activity. The present invention is not limited to SEQ ID NO: 2 in so far as an amino acid sequence having a comparable function to that of SEQ ID NO: 2 is encoded. “Amino acid change” involves one or more amino acids, preferably 1-20, more preferably 1-10, most preferably 1-5 amino acids.

The amino acid sequence encoded by DES1 has an identity of at least about 70%, preferably about 80% or more, more preferably 90% or more, still more preferably 95% or more, and most preferably 98% or more to the amino acid sequence of SEQ ID NO: 2.

The percent amino acid identity may be determined by visual inspection and mathematical calculation. Alternatively, the percent identity of two protein sequences can be determined by comparing sequence information using the GAP computer program, based on the algorithm of Needleman, S. B, and Wunsch, C. D. (J. Mol. Biol., 48: 443-453, 1970), available from the University of Wisconsin Genetics Computer Group (UWGCG). The preferred default parameters for the GAP program include: (1) a scoring matrix, blosum62, as described by Henikoff, S and Henikoff, J. G. (Proc. Natl. Acad. Sci. USA, 89: 10915-10919, 1992); (2) a gap weight of 12; (3) a gap length weight of 4; and (4) no penalty for end gaps.

Other programs used by those skilled in the art of sequence comparison may also be used. The percent identity can be determined by comparing sequence information using the BLAST program described by Altschul et al. (Nucl. Acids. Res. 25, pp. 3389-3402, 1997), for example. This program is available at the website of National Center for Biotechnology Information (NCBI) or DNA Data Bank of Japan (DDBJ) on the Internet. Various conditions (parameters) for homology searches with the BLAST program are described in detail on the site, and searches are normally performed with default values, though some settings may be appropriately changed.

In the methods of the present invention, sphingoid Δ4-desaturase preferably has the amino acid sequence of SEQ ID NO: 2 or an amino acid sequence at least 70% identical to SEQ ID NO: 2, and sphingoid Δ4-desaturase activity.

It is well known to those skilled in the art that even proteins having the same function may have different amino acid sequences depending on the varieties from which they are derived. DES1 may include such homologs and variants of the nucleotide sequence of SEQ ID NO; 1 so long as they have sphingoid Δ4-desaturase activity. In addition to the human sphingoid Δ4-desaturase protein of SEQ ID NO; 2, the presence of genes encoding proteins showing a similar activity is known in, for example, mouse (M. musculus), drosophila (D. melanogaster), nematode (C. elegans), fission yeast (Schizosaccharomyces pombe), etc. (Non-patent document 10).

The expression “having sphingoid Δ4-desaturase activity” refers to the activity of introducing a double bond into C-4 of dihydrosphingosine to synthesize sphingosine, as shown in FIG. 2 or FIG. 3. Alternatively, it refers to the activity of introducing a double bond into C-4 of dihydroceramide to synthesize ceramide NS. Sphingosine and/or ceramide NS that are not synthesized in the natural metabolic pathway of yeast are synthesized in transformant yeast cells by introducing DES1.

A preferred sphingoid Δ4-desaturase gene of the present invention also includes a nucleic acid capable of hybridizing to the nucleotide sequence of SEQ ID NO: 1 under stringent conditions; e.g., under conditions of moderate or high stringency and having sphingoid Δ4-desaturase activity.

The expression “under stringent conditions” refers to hybridization under conditions of moderate or high stringency. Specifically, conditions of moderate stringency can be readily determined by those having ordinary skill in the art based on, for example, the length of the DNA. The basic conditions are set forth by Sambrook et al. Molecular Cloning: A Laboratory Manual, 3rd Ed., Chapters 6-7, Cold Spring Harbor Laboratory Press, 2001, and include use of a prewashing solution for the nitrocellulose filters 5×SSC, 0.5% SDS, 1.0 mM EDTA (pH 8.0), hybridization conditions of about 50% formamide, 2×SSC to 6×SSC at about 40° C. to 50° C. (or other similar hybridization solution, such as Stark's solution, in about 50% formamide at about 42° C.), and washing conditions of about 40° C. to 60° C., 0.5 to 6×SSC, 0.1% SDS. Preferably, conditions of moderate stringency Include hybridization conditions (and washing conditions) of 6×SSC at about 50° C. Conditions of high stringency can also be readily determined by the skilled artisan based on, for example, the length of the DNA

Generally, such conditions include hybridization and/or washing at higher temperatures and/or lower salt concentrations than in the conditions of moderate stringency (e.g., hybridization in 6×SSC to 0.2×SSC, preferably 6×SSC, more preferably 2×SSC, most preferably 0.2×SSC at about 65° C.), and are defined to involve hybridization conditions as above and washing in 0.2×SSC, 0.1% SDS at about 65° C. to 68° C. SSPE (1×SSPE=0.15 M NaCl, 10 mM NaH₂PO₄, and 1.25 mM EDTA, pH 7.4) can be substituted for SSC (1×SSC=0.15 M NaCl and 15 mM sodium citrate) for use as hybridization and washing buffers, and washing is continued for 15 minutes after completion of hybridization.

As known by those skilled in the art and as further described below, it should be understood that the washing temperature and the washing salt concentration can be adjusted as desired to achieve a desirable degree of stringency by applying basic principles governing hybridization reaction and duplex stability (see e.g., Sambrook et al., 2001). When a nucleic acid is to be hybridized to a target nucleic acid of an unknown sequence, the length of the hybrid is assumed to be that of the nucleic acid to be hybridized. When nucleic acids of known sequences are to be hybridized, the length of the hybrid can be determined by aligning the sequences of the nucleic acids and identifying a single or multiple region(s) having optimal sequence complementarity. The hybridization temperature of a hybrid estimated to have a length of less than 50 bp must be 5-25° C. lower than the melting temperature (Tₘ) of the hybrid, where Tₘ is determined by the equation below. For hybrids having a length of less than 18 bp, Tₘ (° C.)=2 (the number of A+T bases)+4 (the number of G+C bases). For hybrids having a length of 18 bp or more, Tₘ=81.5° C.+16.6 (log₁₀[Na⁺])+41 (mole fraction [G+C])−0.63 (% formamide)−500/n, where N is the number of bases in the hybrid, and [Na⁺] is the sodium ion concentration In the hybridization buffer ([Na⁺] in 1×SSC=0.165 M). Preferably, such hybridizing nucleic acids each have a length of at least 8 nucleotides (or more preferably at least 16 nucleotides, or at least 20 nucleotides, or at least 25 nucleotides, or at least 30 nucleotides, or at least 40 nucleotides, or most preferably at least 50 nucleotides), or a length of at least 1% (more preferably at least 25%, or at least 50%, or at least 70%, and most preferably at least 80%) of the length of a nucleic acid to which it hybridizes, and has a sequence identity of at least 50% (more preferably at least 70%, at least 75%, at least 80%, at least 85%, at least 90%, at least 95%, at least 97.5%, or at least 99%, and most preferably at least 99.5%) to a nucleic acid to which it hybridizes. The sequence identity here is determined by comparing the sequences of the nucleic acids to be hybridized when aligned so as to maximize overlap and identity while minimizing sequence gaps, as described in detail above.

The percent nucleic acid identity can be determined by visual inspection and mathematical calculation. Alternatively, the percent identity of two nucleic acid sequences can be determined by visual inspection and mathematical calculation, or more preferably, the comparison is made by comparing sequence information using a computer program. An exemplary, preferred computer program is the Genetics Computer Group (GCG; Madison, Wis.) Wisconsin package version 10.0 program, “GAP” (Devereux et al., 1984, Nucl. Acids Res. 12: 387). This “GAP” program can be used to compare not only two nucleic acid sequences but also two amino acid sequences or a nucleic acid sequence and an amino acid sequence. The preferred default parameters for the “GAP” program include (1) The GCG Implementation of a unary comparison matrix (containing a value of 1 for identities and 0 for non-identities) for nucleotides, and the weighted amino acid comparison matrix of Gribskov and Burgess, Nucl. Acids Res. 14: 6745, 1986 as described by Schwartz and Dayhoff, eds., “Atlas of Polypeptide Sequence and Structure”, National Biomedical Research Foundation, pp. 353-358, 1979; or other comparable comparison matrices; (2) a penalty of 30 for each gap and an additional penalty of 1 for each symbol in each gap for amino acid sequences, or penalty of 50 for each gap and an additional penalty of 3 for each symbol in each gap for nucleotide sequences; (3) no penalty for end gaps; and (4) no maximum penalty for long gaps. Other programs used by those skilled in the art of sequence comparison can also be used, such as, for example, the BLASTN program version 2.2.7, available for use via the National Library of Medicine website: http://www.ncbi.nlm.nih.gov/blast/b12seq/bls.html, or the UW-BLAST 2.0 algorithm. Standard default parameter settings for UW-BLAST 2.0 are described at the following Internet site: http://blast.wustl.edu. In addition, the BLAST algorithm uses the BLOSUM62 amino acid scoring matrix, and optional parameters that can be used are as follows: (A) inclusion of a filter to mask segments of the query sequence that have low compositional complexity (as determined by the SEG program of Wootton and Federhen (Computers and Chemistry, 1993); also see Wootton and Federhen, 1996, Analysis of compositionally biased regions in sequence databases, Methods Enzymol. 266: 554-71) or segments consisting of short-periodicity internal repeats (as determined by the XNU program of Claverie and States (Computers and Chemistry, 1993)), and (B) a statistical significance threshold for reporting matches against database sequences, or E-score (the expected probability of matches being found merely by chance, according to the stochastic model of Karlin and Altschul, 1990: if the statistical significance ascribed to a match is greater than this E-score threshold, the match will not be reported); preferred E-score threshold values are 0.5, or in order of increasing preference, 0.25, 0.1, 0.05, 0.01, 0.001, 0.0001, 1e-5, 1e-10, 1e-15, 1e-20, 1e-25, 1e-30, 1e-40, 1e-50, 1e-75, or 1e-100.

The sphingoid Δ4-desaturase gene (DES1) of the present invention also includes a nucleic acid having a nucleotide sequence different from that of SEQ ID NO: 1 by deletion, addition or substitution of one or more nucleotides, but still encoding a protein having sphingoid Δ4-desaturase activity The number of nucleotides deleted, inserted or substituted is not limited so long as a protein having sphingoid Δ4-desaturase activity is encoded, but is preferably 1 to several thousands, more preferably 1 to 1,000, more preferably 1 to 500, still more preferably 1 to 200, most preferably 1 to 100.

A given amino acid may be replaced, for example, by a residue having similar physiochemical characteristics. Examples of such conservative substitutions include changes from one aliphatic residue to another, such as changes from one to another of Ile, Val, Leu, or Ala; changes from one polar residue to another, such as Lys to Arg, Glu to Asp, or Gin to Asn; or changes from one aromatic residue to another, such as changes from one to another of Phe, Trp, or Tyr. Other well-known conservative substitutions include, for example, changes between entire regions having similar hydrophobic characteristics. Those skilled in the art can introduce desired deletions, insertions or substitutions by well-known gene engineering techniques using, for example, site-specific mutagenesis as described in Sambrook et al. (2001), supra.

Yeast Sphinganine C4-Hydroxylase Gene (SUR2)

The methods of the present invention comprise abolishing the expression of the yeast sphinganine C4-hydroxylase gene (SUR2) by transformation of the yeast cell, as essential feature 2).

SUR2 preferably has, but is not limited to, the amino acid sequence of SEQ ID NO: 6 or an amino acid sequence obtained by deletion, addition or substitution of one or more amino acid residues in SEQ ID NO: 6, and encodes a protein having sphinganine C4-hydroxylase activity.

SUR2 preferably has the nucleotide sequence of SEQ ID NO: 5. This is the nucleotide sequence encoding a yeast sphinganine C4-hydroxylase protein having the amino acid sequence of SEQ ID NO; 6, and it is disclosed in, for example, SGD (Saccharomyces Genome Database, http://www.yeastgenome.org/).

SUR2 preferably encodes the amino acid sequence of SEQ ID NO: 6. However, it may also have an amino acid sequence obtained by deletion, addition or substitution of one or more amino acid residues. It is intended to encompass any homologous protein so long as it has sphinganine C4-hydroxylase activity. The present invention is not limited to SEQ ID NO: 6 so long as an amino acid sequence having a comparable function to that of SEQ ID NO: 6 is encoded. “Amino acid change” involves one or more amino acids, preferably 1-20, more preferably 1-10, most preferably 1-5 amino acids.

The expression “having sphinganine C4-hydroxylase activity” refers to the activity of introducing a hydroxyl group into C-4 of dihydrosphingosine to synthesize phytosphingosine, as shown in FIG. 2 or FIG. 3. Alternatively, it refers to the activity of introducing a hydroxyl group into C-4 of dihydroceramide to synthesize phytoceramide. In the present invention, the synthesis of phytosphingosine and/or phytoceramide that are synthesized in the natural metabolic pathway of yeast is partially or completely inhibited by partially or completely abolishing the expression of SUR2 by transformation of the yeast cell. Sphingosine and/or ceramide NS can be efficiently synthesized by suppressing the expression of the SUR2 gene and expressing the DES1 gene.

The amino acid sequence encoded by SUR2 has an identity of at least about 70%, preferably about 80% or more, more preferably 90% or more, still more preferably 95% or more, most preferably 98% or more to the amino acid sequence of SEQ ID NO: 6.

A preferred yeast sphinganine C4-hydroxylase gene (SUR2) of the present invention also includes a nucleic acid capable of hybridizing to the nucleotide sequence of SEQ ID NO; 5 under stringent conditions; e.g., under conditions of moderate or high stringency and having yeast sphinganine C4-hydroxylase activity.

The yeast sphinganine C4-hydroxylase gene (SUR2) of the present invention also includes a nucleic acid having a nucleotide sequence different from that of SEQ ID NO: 5 by deletion, addition or substitution of one or more nucleotides, but still encoding a protein having sphinganine C4-hydroxylase activity.

Common matters such as “deletion, addition or substitution of amino acids and/or nucleotides”, “percent identity of amino acids and/or nucleotides”, hybridization “under stringent conditions” are as described above for DES1.

Yeast Sphingolipid α-Hydroxylase Gene (SCS7)

The methods of the present invention may further comprise abolishing the expression of the yeast sphingolipid α-hydroxylase gene (SCS7) by transformation of the yeast cell. SCS7 has the activity of adding a hydroxyl group to the α-carbon of a fatty acid amide-linked to the sphingoid base of phytoceramide, dihydroceramide, and ceramide NS to synthesize Cer(AP), Cer(ASa), and Cer(AS), respectively, in FIG. 3 or FIG. 7, for example. Even if desired dihydroceramide or ceramide NS is synthesized, it is further hydroxylated by the presence of SCS7. Thus, the present invention preferably comprises abolishing the expression of SCS7.

SCS7 preferably has, but not limited to, the amino acid sequence of SEQ ID NO: 8 or an amino acid sequence obtained by deletion, addition or substitution of one or more amino acid residues in SEQ ID NO: 8, and encodes a protein having sphingolipid α-hydroxylase activity.

SCS7 preferably has the nucleotide sequence of SEQ ID NO: 7. This is the nucleotide sequence encoding a yeast sphingolipid α-hydroxylase protein having the amino acid sequence of SEQ ID NO: 8, and it is disclosed in e.g., SGD (Saccharomyces Genome Database, http://Www.yeastgenome.org/).

SCS7 preferably encodes the amino acid sequence of SEQ ID NO: 8. However, it may also have an amino acid sequence obtained by deletion, addition or substitution of one or more amino acid residues. It is intended to encompass any homologous protein so long as it has sphingolipid α-hydroxylase activity. The present invention is not limited to SEQ ID NO: 8 so long as an amino acid sequence having a comparable function to that of SEQ ID NO: 8 is encoded. “Amino acid change” involves one or more amino acids, preferably 1-20, more preferably 1-10, most preferably 1-5 amino acids.

The expression “having sphingolipid α-hydroxylase activity” refers to the activity of adding a hydroxyl group to the α-carbon of a fatty acid amide-linked to the sphingoid base of phytoceramide, dihydroceramide, and ceramide NS to synthesize Cer(AP), Cer(ASa), and Cer(AS), respectively, as shown in FIG. 7, for example. In the present invention, undesirable hydroxylation of dihydroceramide or ceramide NS is inhibited by partially or completely abolishing the expression of SCS7, preferably by transformation of the yeast cell.

The amino acid sequence encoded by SCS7 has an identity of at least about 70%, preferably about 80% or more, more preferably 90% or more, still more preferably 95% or more, most preferably 98% or more to the amino acid sequence of SEQ ID NO: 8.

A preferred yeast sphingolipid α-hydroxylase gene (SCS7) of the present invention also includes a nucleic acid capable of hybridizing to the nucleotide sequence of SEQ ID NO: 7 under stringent conditions, e.g., under conditions of moderate or high stringency and having yeast sphingolipid α-hydroxylase activity.

The yeast sphingolipid α-hydroxylase gene (SCS7) of the present invention also includes a nucleic acid having a nucleotide sequence different from that of SEQ ID NO: 7 by deletion, addition or substitution of one or more nucleotides, but still encoding a protein having sphingolipid α-hydroxylase activity.

Common matters such as “deletion, addition or substitution of amino acids and/or nucleotides”, “percent identity of amino acids and/or nucleotides”, hybridization “under stringent conditions” are as described above for DES1.

Yeast Alkaline Dihydroceramidase Gene (YDC1)

The methods of the present invention may further comprise abolishing the expression of the yeast alkaline dihydroceramidase gene (YDC1) by transformation of the yeast cell.

YDC1 encodes a protein having the activity of degrading dihydroceramide to synthesize dihydrosphingosine, as shown in FIG. 1-FIG. 3, for example. YDC1 activity promotes so to speak a reverse reaction to ceramide synthesis to reduce dihydroceramide as a precursor for ceramide NS synthesis. Thus, the present invention preferably comprises abolishing the expression of YDC1.

YDC1 preferably has, but is not limited to, the amino acid sequence of SEQ ID NO: 10 or an amino acid sequence obtained by deletion, addition or substitution of one or more amino acid residues in SEQ ID NO: 10, and encodes a protein having alkaline dihydroceramidase activity.

YDC1 preferably has the nucleotide sequence of SEQ ID NO: 9. This is the nucleotide sequence encoding a yeast alkaline dihydroceramidase protein having the amino acid sequence of SEQ ID NO: 10, and it is disclosed in, for example, SGD (Saccharomyces Genome Database, http://www.yeastgenome.org/).

YDC1 preferably encodes the amino acid sequence of SEQ ID NO: 10. However, it may also have an amino acid sequence obtained by deletion, addition or substitution of one or more amino acid residues. It is intended to encompass any homologous protein so long as it has alkaline dihydroceramidase activity. The present invention is not limited to SEQ ID NO: 10 so long as an amino acid sequence having a comparable function to that of SEQ ID NO: 10 is encoded. “Amino acid change” involves one or more amino acids, preferably 1-20, more preferably 1-10, and most preferably 1-5 amino acids.

The expression “having alkaline dihydroceramidase activity” refers to the activity of degrading dihydroceramide to synthesize dihydrosphingosine, as shown in FIG. 1-FIG. 3, for example. In the present invention, undesirable degradation of dihydroceramide is inhibited by partially or completely abolishing the expression of YDC1, preferably by transformation of the yeast cell.

The amino acid sequence encoded by YDC1 has an identity of at least about 70%, preferably about 80% or more, more preferably 90% or more, still more preferably 95% or more, most preferably 98% or more to the amino acid sequence of SEQ ID NO: 10.

A preferred yeast alkaline dihydroceramidase gene (YDC1) of the present invention also includes a nucleic acid capable of hybridizing to the nucleotide sequence of SEQ ID NO: 9 under stringent conditions. e.g., under conditions of moderate or high stringency and having yeast alkaline dihydroceramidase activity.

The yeast alkaline dihydroceramidase gene (YDC1) of the present invention also includes a nucleic acid having a nucleotide sequence different from that of SEQ ID NO: 9 by deletion, addition or substitution of one or more nucleotides, but still encoding a protein having alkaline dihydroceramidase activity.

Common matters such as “deletion, addition or substitution of amino acids and/or nucleotides”, “percent identity of amino acids and/or nucleotides”, hybridization “under stringent conditions” are as described above for DES1.

Yeast Inositol Phosphosphingolipid Phospholipase C Gene (ISC1)

The methods of the present invention may further comprise enhancing the expression of the yeast inositol phosphosphingolipid phospholipase C gene (ISC1) by transformation of the yeast cell.

ISC1 encodes a protein having the activity of synthesizing dihydroceramide from inositol phosphate ceramide (IPC) as shown in FIG. 1 and FIG. 2, for example. ISC1 activity increases dihydroceramide as a precursor for ceramide NS synthesis. ISC1 is a gene naturally occurring in yeast cells, but the synthesis of human ceramide can be promoted by introducing this gene to enhance its expression by transformation of the yeast cell.

ISC1 preferably has, but is not limited to, the amino acid sequence of SEQ ID NO: 4 or an amino acid sequence obtained by deletion, addition or substitution of one or more amino acid residues in SEQ ID NO: 4, and encodes a protein having inositol phosphosphingolipid phospholipase C activity.

ISC1 preferably has the nucleotide sequence of SEQ ID NO: 3. This is the nucleotide sequence encoding a yeast inositol phosphosphingolipid phospholipase C protein having the amino acid sequence of SEQ ID NO: 4, and it is disclosed in, for example, SGD (Saccharomyces Genome Database, http://www.yeastgenome.org/).

ISC1 preferably encodes the amino acid sequence of SEQ ID NO: 4. However, it may also have an amino acid sequence obtained by deletion, addition or substitution of one or more amino acid residues. It is intended to encompass any homologous protein so long as it has inositol phosphosphingolipid phospholipase C activity. The present invention is not limited to SEQ ID NO: 4 so long as an amino acid sequence having a comparable function to that of SEQ ID NO: 4 is encoded, “Amino acid change” involves one or more amino acids, preferably 1-20, more preferably 1-10, and most preferably 1-5 amino acids.

The expression “having inositol phosphosphingolipid phospholipase C activity” refers to the activity of synthesizing dihydroceramide from inositol phosphate ceramide (IPC), as shown in FIG. 1 and FIG. 2. In the present invention, dihydroceramide as a precursor for ceramide NS synthesis is increased to promote the synthesis of human ceramide by enhancing the expression of ISC1, preferably by transformation of the yeast cell.

The amino acid sequence encoded by ISC1 has an identity of at least about 70%, preferably about 80% or more, more preferably 90% or more, still more preferably 95% or more, and most preferably 98% or more to the amino acid sequence of SEQ ID NO: 4.

A preferred yeast inositol phosphosphingolipid phospholipase C gene (ISC1) of the present invention also includes a nucleic acid capable of hybridizing to the nucleotide sequence of SEQ ID NO: 3 under stringent conditions, e.g., under conditions of moderate or high stringency and having yeast inositol phosphosphingolipid phospholipase C activity.

The yeast inositol phosphosphingolipid phospholipase C gene (ISC1) of the present invention also includes a nucleic acid having a nucleotide sequence different from that of SEQ ID NO: 3 by deletion, addition or substitution of one or more nucleotides, but still encoding a protein having inositol phosphosphingolipid phospholipase C activity.

Common matters such as “deletion, addition or substitution of amino acids and/or nucleotides”, “percent identity of amino acids and/or nucleotides”, hybridization “under stringent conditions” are as described above for DES1.

In a more preferred embodiment, the present invention provides a method for producing human ceramide in a yeast cell, comprising:

1) introducing the sphingoid Δ4-desaturase gene (DES1) by transformation of the yeast cell;

2) abolishing the expression of the yeast sphinganine C4-hydroxylase gene (SUR2) by transformation of the yeast cell;

3) abolishing the expression of the yeast sphingolipid α-hydroxylase gene (SCS7) by transformation of the yeast cell;

4) abolishing the expression of the yeast alkaline dihydroceramidase gene (YDC1) by transformation of the yeast cell; and

5) enhancing the expression of the yeast inositol phosphosphingolipid phospholipase C gene (ISC1) by transformation of the yeast cell.

Methods for Introducing and Expressing Genes by Transformation of Yeast

In the present invention, the expression of DES1 and the enhanced expression of ISC1 in the yeast cell can be performed by any known method. Preferably, the method comprises transforming a host yeast cell with an expression vector containing DES1 and/or ISC1, and cultivating the transformant yeast cell under conditions allowing the expression of the nucleic acid.

The yeast species that can be used in the methods of the present invention are preferably, but are not limited to, yeast species of the genus Saccharomyces. Saccharomyces cerevisiae, Saccharomyces pastorianus, Saccharomyces bayanus, and Saccharomyces kluyveri are more preferred. Budding yeast species including the genus Saccharomyces have been analyzed most extensively for ceramide synthesis and metabolism at the genetic level. Thus, they can be used to rapidly optimize the methods for producing human ceramide according to the present invention. Moreover, yeast cells are easy to culture and have been traditionally used for food manufacturing. In addition, they can be used to establish a method for extracting/purifying large amounts of ceramides conveniently, safely and inexpensively.

In the present invention, known yeast expression vectors can be used to introduce and express genes. In the examples below, known gene expression vectors for yeast pRS series (p4XX) (Mumberg et al., Gene, 156, 119, 1995) and pYE22m (Ashikari et al., Appl Microbiol Biotechnol, 30, 515, 1989) were used.

Any of multicopy (YEp), single copy (YCp), and chromosome integration (YIp) vectors can be used for introduction into yeast. For example, a number of expression vectors for yeast are known to those skilled in the art and can be used in the methods of the present invention, including YEp vectors such as YEp24 (J. R. Broach et al., Experimental Manipulation of Gene Expression, Academic Press, New York, 83, 1983), YCp vectors such as YCp50 (M. D. Rose et al., gene, 60, 237, 1987), and YIp vectors such as YIp5 (K. Struhl et al., Proc. Natl. Acad. Sci. USA, 76, 1035, 1979).

In addition to each gene of interest, expression vectors can typically contain a selectable marker and an origin of replication for proliferation in host cells. Vectors also optionally contain a transcription or translation regulatory sequence preferably derived from yeast fused to a nucleic acid of the present invention.

Examples of regulatory sequences include transcriptional promoters, operators, or enhancers, an mRNA ribosomal binding site, and appropriate sequences which control the initiation and termination of transcription and translation. Nucleotide sequences are operably linked to a regulatory sequence when the regulatory sequence is functionally associated with the DNA sequences. Thus, a promoter nucleotide sequence is operably linked to a DNA sequence if the promoter nucleotide sequence controls the transcription of the DNA sequence. An origin of replication that confers the ability to replicate in a host cell, and a selection gene by which transformants are identified are generally incorporated into expression vectors. As for selectable markers, those commonly used can be routinely used. Examples are genes resistant to antibiotics such as tetracycline, ampicillin, kanamycin, neomycin, hygromycin or spectinomycin and auxotrophic genes such as HIS3, TRP1.

Yeast vectors will often contain an origin of replication sequence derived from the 2μ yeast plasmid, an autonomous replication sequence (ARS), a promoter region, a sequence for polyadenylation, a sequence for transcription termination, and a selectable marker gene.

Vectors can be conveniently prepared by routine fusion of a desired gene to a recombination vector available in the art (e.g. plasmid DNA). Methods for integrating a DNA fragment of a gene into a vector such as a plasmid are described in, for example, Sambrook, J., and Russell, D. W. (2001). Molecular Cloning: A Laboratory Manual, 3rd ed. (New York: Cold Spring Harbor Laboratory Press). Commercially available ligation kits (e.g. available from TAKARA) can be conveniently used.

Methods for introducing a plasmid into a host cell include calcium phosphate or calcium chloride/rubidium chloride transfection, electroporation, electroinjection, chemical treatment with PEG or the like, the use of a gene gun, etc. described in Sambrook, J. et al. (2001) (supra.).

Methods for Abolishing Each Gene by Transformation of Yeast

The methods of the present invention comprise abolishing the expression of the yeast sphinganine C4-hydroxylase gene (SUR2) by transformation of the yeast cell.

In preferred embodiments, the methods of the present invention further comprise either one or a combination of:

abolishing the expression of the yeast sphingolipid α-hydroxylase gene (SCS7) by transformation of the yeast cell; and

abolishing the expression of the yeast alkaline dihydroceramidase gene (YDC1) by transformation of the yeast cell.

As used herein, the expression “abolishing the expression of each gene” means that the protein activity encoded by each gene is not produced. Any abolishment is included in the scope of the present invention to achieve the purposes in the methods of the present invention, so long as the protein activity encoded by the gene is not exerted eventually, such as disrupting the gene on the genome of a yeast cell, inhibiting the transcription of the gene, inhibiting the translation of the gene into a protein, or inhibiting activity even if it is translated into a protein. Abolishment may be partial or complete. Typically, each gene on the genome of a mother cell is disrupted to partially or completely delete the gene.

The expression of the SUR2, SCS7 and YDC1 genes can be abolished by known methods.

For example, a DNA fragment containing upstream and downstream nucleotide sequences of each gene fused to a selectable marker was used to delete the gene by homologous recombination with the natural genome sequence of yeast in the examples herein below.

Disruption of a gene can be performed by addition or deletion of one or more nucleotides in a region responsible for the expression of a gene product in the target gene, such as a coding region or a promoter region, or by entire deletion of these regions. Such methods for gene disruption can be found in known publications (e.g., see Yeast 10, 1793 (1994), Yeast 15, 1541 (1999), Proc. Natl. Acad. Sci. USA, 76, 4951 (1979), Methods in Enzymology, 101, 202 (1983), etc.).

In addition to gene disruption, other methods for suppressing the expression of each gene for the purposes of the present invention include antisense methods (e.g., see Hirajima and Inoue: New Biochemistry Experiment Course 2 Nucleic acid, IV. Gene Replication and Expression (Japanese Biochemical Society Ed., Tokyo Kagaku Dozin Co., Ltd.) pp. 319-347, 1993, etc.), RNAi methods (see Domestic announcement No. 2002-516062 of PCT application; US Patent Laid-Open Publication No. 2002/086356A; Nature Genetics, 24 (2), 180-183, 2000, etc.), ribozyme methods (see FEES Lett. 228: 228, 1988; FEBS Lett. 239: 285, 1988; Nucl. Acids. Res. 17: 7059, 1989, etc.), cosuppression (e.g., see Smyth D R: Curr. Biol. 7: R793, 1997, Martienssen R: Curr. Biol, 6: 810, 1996, etc.), etc.

Methods for Verifying Ceramide Synthesis

The human ceramide (ceramide NS) produced by the methods of the present invention can be extracted/purified by using known methods. The methods of the present invention allow large-scale culture and convenient and rapid extraction/purification of the ceramide because yeast cells are used. One of the preferred embodiments for extraction/purification used in the examples is shown in FIG. 4.

The purified ceramide can be identified by using known methods for analyzing sphingoid bases. Analytical methods include e.g., TLC, HPLC, mass spectrometry (e.g., LC-MS, LC-MS/MS, FT-MS), etc.

### ADVANTAGES OF THE INVENTION

According to the methods for producing ceramide using yeast transformants of the present invention, human ceramide highly functional on the human skin can be inexpensively produced.

### BRIEF EXPLANATION OF THE DRAWINGS

FIG. 1 shows the synthetic/metabolic pathways for sphingolipids in yeast and higher animal cells.

FIG. 2 shows an overview of a preferred embodiment of a method for producing human ceramide in yeast cells according to the present invention.

FIG. 3 shows the molecular species of sphingoid bases and ceramides and the structural formulae thereof in yeast and higher animals.

FIG. 4 schematically shows steps of the present invention from cultivation of yeast cells to analyses by TLC and HPLC.

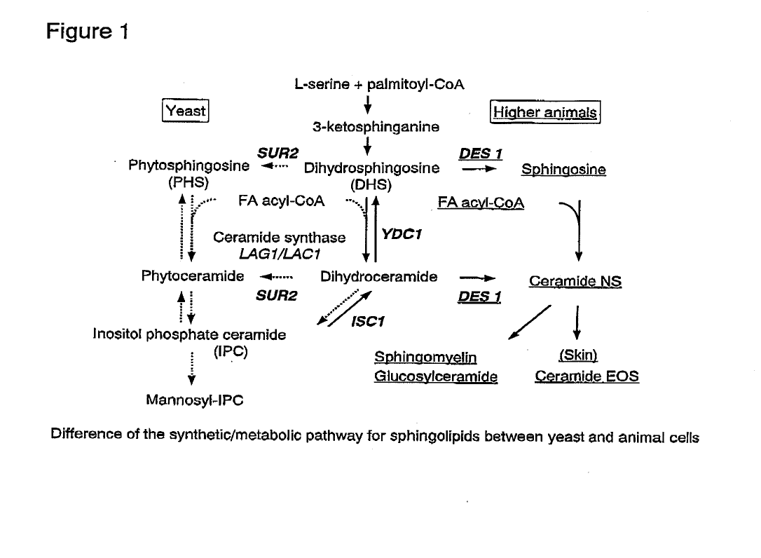

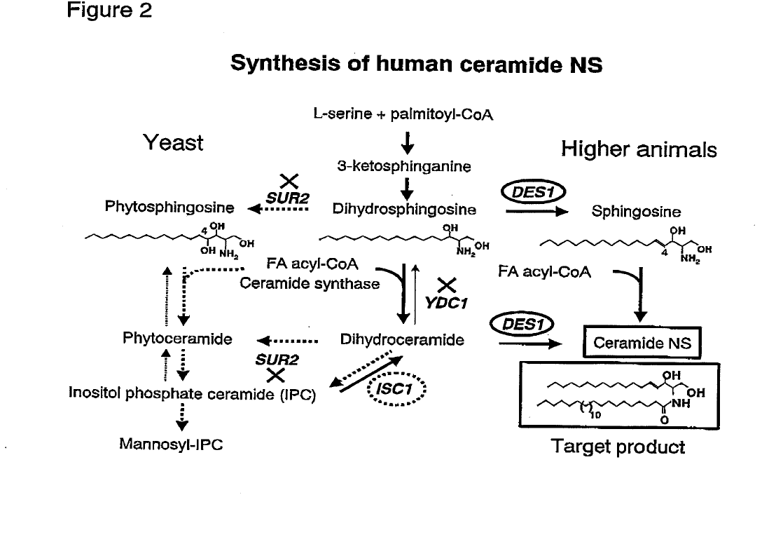

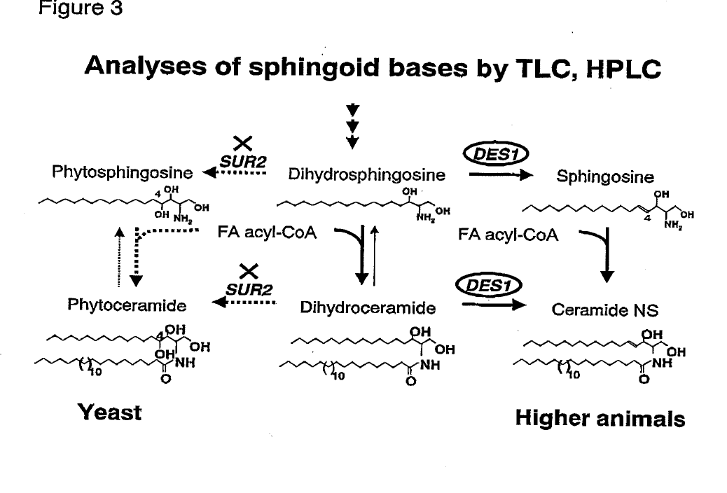

FIG. 5 shows the results of the identification of the molecular species of ceramides by LC-MS analysis in a yeast SUR2/SCS7 double disruption strain expressing the human DES1 gene.
Left panels show from top to bottom:
a total cation chromatogram of the ceramide region of the yeast SUR2/SCS7 double disruption strain expressing the human DES1 gene;
a chromatogram generated only at m/z 680 (peak RT13.35) in the same strain;
a chromatogram generated only at m/z 678 (peak RT13.69) in the same strain; and
a cation chromatogram of standard ceramide NS.
Right panels show from top to bottom:
a mass spectrum at a peak of m/z 680; and
a mass spectrum at a peak of m/z 678.
It could be verified that ceramide NS (Cer(NS) (Mw 678)) had been synthesized in the yeast SUR2/SCS7 double disruption strain expressing the human DES1 gene.

FIG. 6 shows the results of analyses of sphingoid bases by TLC and HPLC in a SUR2 disruption strain.
Sample 1: phytosphingosine standard,
Sample 2: dihydrosphingosine standard,
Sample 3; sphingosine standard,
Sample 4: sphingoid base extracted from wild-type yeast,
Sample 5: sphingoid base extracted from the SUR2 disruption strain.
Panels show from left to right:
TLC observed under visible light (DNP-derivatized sphingoid bases are observed as yellow spots),
TLC observed under UV radiation (DNP-derivatized sphingoid bases are observed as dark blue spots), and
HPLC chromatograms.
The sphingoid base extracted from the SUR2 disruption strain (sample 5) was identified as dihydrosphingosine from the location of the HPLC peak agreeing with that of sample 2. In contrast, the sphingoid base extracted from wild-type yeast (sample 4) was identified as phytosphingosine from the location of the HPLC peak agreeing with that of sample 1.

FIG. 7 shows the results of LC-MS analysis of a SUR2/SCS7 double disruption strain (ion chromatogram at m/z 500-800). In the SUR2/SCS7 double disruption strain, accumulation of dihydroceramide (Cer(NSa)) was verified. If DES1 is expressed by this disruption strain, human ceramide will be synthesized.

FIG. 8 shows ceramide levels in the yeast SUR2/SCS7 double disruption strain expressing human DES1 and the yeast SUR2/SCS7/YDC1 triple disruption strain expressing human DES1.

FIG. 9 shows the results of TLC analysis of yeast ceramides using tritiated (³H) D-erythro-dihydrosphingosine and the results of quantification of radioactively labeled ceramides using a Bioimage Analyzer (BAS). The amount of ceramide NS synthesized increased in order from left: sample 1: SUR2/SCS7 double disruption strain; sample 2; yeast SUR2/SCS7 double disruption strain expressing the human DES1 gene; sample 3: yeast SUR2/SCS7/YDC1 triple disruption strain expressing the human DES1; and sample 4: yeast SUR2/SCS7/YDC1 triple disruption strain expressing human DES1 and highly expressing ISC1.

### EXAMPLES

The following examples further illustrate the present invention but are not intended to limit the technical scope of the invention. Those skilled in the art can readily add modifications/changes to the present invention in the light of the description herein, and those modifications/changes are included in the technical scope of the present invention.

### Example 1

### Preparation of a Vector Expressing the Human Sphingoid Δ4-Desaturase Gene (DES1)

Based on the nucleotide sequence of the human sphingoid Δ4-desaturase gene (DES1) in a public database (GenBanK™: accession number AF466375) (SEQ ID NO: 1), primers des1F (SEQ ID NO: 11) and des1R (SEQ ID NO: 12) were prepared.

SEQ ID NO: 11:
5′-CCTTCTCTAGAGGATCCATGGGGAGCCGCGTCTCGCGGGAAGAC-3′
SEQ ID NO: 12:
5′-CCTTCGAATTCCCCGGGCCAGGGGAGCTTCTGAGCATCACTGGTC-
3′.

The primer pair was used to perform PCR with a human cDNA library as a template. The resulting PCR product (about 1.1 kb) was cloned into the gene expression vector for yeast pKO11 (Kamei et al., J. Biol. Chem., 273, 28341, 1998; provided by Dr. K. Tanaka) using BamHI and SmaI sites.

The nucleotide sequence of the clone was determined by the Sanger method to confirm that it was identical to the sequence in the database. The clone was subcloned into the gene expression vector for yeast pRS series (p4XX) (Mumberg et al., Gene, 156, 119, 1995) using BamHI and Xho1 sites.

### Example 2

### Preparation of a Vector Expressing the Yeast (Saccharomyces cerevisiae) Inositol Phosphosphingolipid Phospholipase C Gene (ISC1)

Based on the nucleotide sequence of the yeast (Saccharomyces cerevisiae) inositol phosphosphingolipld phospholipase C gene (ISC1) in a public yeast genome database (SGD (Saccharomyces Genome Database, http://www.yeastgenome.org/)) (SEQ ID NO: 3), primers isc1F (SEQ ID NO: 13) and isc1R (SEQ ID NO: 14) were prepared.

SEQ ID NO 13:
5′-ATGTACAACAGAAAAGACAGAGATG-3′
SEQ ID NO: 14:
5′-AAGGTACCTCATTTCTCGCTCAAGAAAGTT-3′.

The primer pair was used to perform PCR with a routinely prepared yeast genomic DNA as a template. The resulting PCR product (about 1.4 kb) was cloned into the pCR-BluntII-TOPO vector (Invitrogen), and the nucleotide sequence of the clone was determined by the Sanger method (P. Sanger, Science, 214, 1215, 1981) to confirm that it was identical to the sequence in the database.

The clone was subcloned into the gene expression vector for yeast pYE22m (Ashikari et al., Appl Microbiol Biotechnol, 30, 515, 1989) using EcoRI and KpnI sites.

### Example 3

### Preparation of a Disruption Strain of the Yeast Sphinganine C4-Hydroxylase Gene (SUR2)

Based on the nucleotide sequence of the yeast sphinganine C4-hydroxylase gene (SUR2) in a public yeast genome database (SGD (Saccharomyces Genome Database, http://www.yeastgenome.org/)) (SEQ ID NO; 5), primers sur2F (SEQ ID NO: 15) and sur2R (SEQ ID NO: 16) were prepared.

SEQ ID NO: 15:
5′-CTCCGGCTTCTGCGGTTTTTCTTAGTCTTTCCGCACCAATTTTCACA
GGAATTCCCGGGGATCCGG-3′
SEQ ID NO: 16:
5′-GGATAATAAATACAAACGTGGGAAGTCGGAGACATTGCCTTTACCCA
GCAAGCTAGCTTGGCTGCAGG-3′.

The primer pair was used to perform PCR with the plasmid pYDp-L (Berben et al., Yeast, 7, 475, 1991) as a template, thereby giving a PCR product containing a 295-bp upstream region of the SUR2 gene, a selectable marker and a 75-bp downstream region of the SUR2 gene fused together. This PCR product was transformed into the strain FK113 (MATa, ura3, his3, leu2, lys2, trp1, bar1-1), and the transformants were selected in an auxotrophic medium to give a SUR2 gene disruption strain.

The disruption of the SUR2 gene was confirmed by PCR using confirmation primers designed to be amplified into fragments of different lengths depending on whether the gene is normal or disrupted (SEQ ID NOs: 17 and 18).

SEQ ID NO: 17:
5′-CTCCGGCTTCTGCGGTTTTTCTTAGTCTTTC-3′
SEQ ID NO: 18:
5′-GGAAGTCGGAGACATTGCCTTTACCCAG-3′.

### Example 4

### Preparation of a Double Disruption Strain of the Yeast SUR2 and Yeast Sphingolipid α-Hydroxylase (SCS7) Genes

Based on the nucleotide sequence of the yeast sphingolipid α-hydroxylase gene (SCS7) in a public yeast genome database (SGD (Saccharomyces Genome Database, http://www.yeastgenome.org/)) (SEQ ID NO: 7), primers scs7up280F (SEQ ID NO: 19) and scs7up280R (SEQ ID NO: 20), and scs7down280F (SEQ ID NO: 21) and scs7down280R (SEQ ID NO; 22) were prepared.

SEQ ID NO: 19:
5′-CGAATTCAGCCGAAAACAGTCTTGCTT-3′
SEQ ID NO: 20:
5′-ACGAGGCTGGGATCCGCTTACCACCGCTTTTAGTGC-3′
SEQ ID NO: 21:
5′-GGTGGTAAGCGGATCCCAGCCTCGTCCAAAATTGTC-3′
SEQ ID NO: 22:
5′-CGAATTCTTGCCAACCTGATCTGTGAA-3′.

The primer pairs were used to perform PCR with a routinely prepared yeast genomic DNA as a template to give PCR products corresponding to an upstream region of about 280 bp and a downstream region of about 280 bp of the SCS7 gene, respectively. The primers scs7up280R and scs7down280F have complementary sequences and contain a BamHI site at the boundary between the upstream and downstream regions of the SCS7 gene. The PCR products corresponding to the upstream and downstream regions of about 280 bp of the SCS7 gene were mixed to perform PCR using the primers scs7up280F and scs7down280R, thereby giving a PCR product containing the upstream and downstream regions of the SCS7 gene flanking the BamHI site. This product was cloned into a vector obtained by self-ligation of pUC19 that had been digested with BamHI and then blunt-ended with T4 DNA polymerase, using EcoRI sites contained in the primers scs7up280F and scs7down280R. The nucleotide sequence of the clone was determined by the Sanger method (F. Sanger, Science, 214, 1215, 1981) to confirm that it was identical to the sequences of the upstream and downstream regions of SCS7 in the database.

The plasmid pYDp-U (Berben et al., Yeast, 7, 475, 1991) was digested with BamHI, and a fragment of about 1.1 kb containing the URA3 gene was inserted into the BamHI site of the plasmid containing the upstream and downstream regions of the SCS7 gene.

A fragment obtained by digesting the resulting plasmid containing the URA3 gene flanked by the upstream and downstream regions of the YDC1 gene with EcoRI was routinely transformed into the SUR2 gene disruption strain obtained in Example 3 above. Screening on complete minimal plate lacking uracil (SC-Ura) gave a SUR2/SCS7 double disruption strain. To allow for the subsequent plasmid introduction, the SUR2/SCS7 double disruption strain was plated on minimal plate containing 0.1% 5-fluoroorotic acid to select a strain having lost uracil auxotrophy.

### Example 5

### Preparation of a Triple Disruption Strain of the Yeast SUR2, Yeast Alkaline Dihydroceramidase (YDC1), and SCS7 Genes

Based on the nucleotide sequence of the yeast alkaline dihydroceramidase gene (YDC1) in a public yeast genome database (SGD (Saccharomyces Genome Database, http://www.yeastgenome.org/)) (SEQ ID NO: 9), primers ydc1up280F (SEQ ID NO: 23) and ydc1up280R (SEQ ID NO: 24), and ydc1down250F (SEQ ID NO: 25) and ydc1down250R (SEQ ID NO: 26) were prepared.

SEQ ID NO: 23:
5′-CGAATTCCCCAGAGGCAAAGATGTTA-3′
SEQ ID NO: 24:
5′-TGGATGGCACGGATCCGAAAGGCACACCTGTCATTATGG-3′
SEQ ID NO: 25:
5′-TGTGCCTTTCGGATCCGTGCCATCCATTTGAATC-3′
SEQ ID NO: 26:
5′-CGAATTCCTTTTATGATGGGAGTAACTGCT-3′.

The primer pairs were used to perform PCR with a routinely prepared yeast genomic DNA as a template, thereby giving PCR products corresponding to an upstream region of about 280 bp and a downstream region of about 250 bp of the YDC1 gene, respectively. The primers ydc1up280R and ydc1down250F have complementary sequences and contain a BamHI site at the boundary between the upstream and downstream regions of the YDC1 gene.

The PCR products corresponding to the upstream region of about 280 bp and downstream region of about 250 bp of the YDC1 gene were mixed to perform PCR using the primers ydc1up280F and ydc1down250R, thereby giving a PCR product containing the upstream and downstream regions of the YDC1 gene flanking the BamHI site. This product was cloned into a vector obtained by self-ligation of pUC19 that had been digested with BamHI and then blunt-ended with T4 DNA polymerase, by use of EcoRI sites contained in the primers ydc1up280F and ydc1down250R. The nucleotide sequence of the clone was determined by the Sanger method (F. Sanger, Science, 214, 1215, 1981) to confirm that it was identical to the sequences of the upstream and downstream regions of YDC1 in the database.

The plasmid pYDp-H (Berben et al., Yeast, 7, 475, 1991) was digested with BamHI, and a fragment of about 1.2 kb containing the HIS3 gene was inserted into the BamHI site of the plasmid containing the upstream and downstream regions of the YDC1 gene.

A fragment obtained by digesting the resulting plasmid containing the HIS3 gene flanked by the upstream and downstream regions of the YDC1 gene with EcoRI was routinely transformed into the SUR2 gene disruption strain. Screening on complete minimal plate lacking histidine (SC-His) gave a SUR2/YDC1 double disruption strain.

Based on the nucleotide sequence of the yeast sphingolipid α-hydroxylase (SCS7) in a public yeast genome database (SGD (Saccharomyces Genome Database, http://www.yeastgenome.org/)) (SEQ ID NO: 7), primers scs7up280F (SEQ ID NO: 19) and scs7up280R_G418 (SEQ ID NO: 27), and scs7down280F_G418 (SEQ ID NO: 28) and scs7down280R (SEQ ID NO: 22) were then prepared.

SEQ ID NO: 19:
5′-CGAATTCAGCCGAAAACAGTCTTGCTT-3′
SEQ ID NO: 27:
5′-CTCCATGTCGCTTACCACCGCTTTTAGTGC-3′
SEQ ID NO: 28:
5′-CGCTATACTGCAGCCTCGTCCAAAATTGTCA-3′
SEQ ID NO: 22:
5′-CGAATTCTTGCCAACCTGATCTGTGAA-3′.

The primer pairs were used to perform PCR with a routinely prepared yeast genomic DNA as a template, thereby giving PCR products corresponding to an upstream region of about 280 bp and a downstream region of about 280 bp of the SCS7 gene, respectively.

Primers G418PCR2F (SEQ ID NO: 29) and G418PCR2R (SEQ ID NO: 30) were also prepared and used to perform PCR with the plasmid pFA6a-kanMX4 (EMBL AJ002680) as a template, thereby giving a PCR product containing a kanamycin and geneticin (G418) resistance gene.

SEQ ID NO: 29:
5′-GTGGTAAGCGACATGGAGGCCCAGAATACC-3′
SEQ ID NO: 30:
5′-GACGAGGCTGCAGTATAGCGACCAGCATTCA-3′

The primers scs7up280R_G418 (SEQ ID NO: 27) and G418PCR2F (SEQ ID NO: 29), and the primers scs7down280F_G418 (SEQ ID NO: 28) and G418PCR2R (SEQ ID NO: 30) have complementary sequences, and the three PCR products were mixed to perform PCR using the primers scs7up280F and scs7down280R, thereby giving a PCR product containing the 280-bp upstream region of the SCS7 gene, the kanamycin and G418 resistance gene and the 280-bp downstream region of the SCS7 gene fused together. The nucleotide sequence of the clone was determined by the Sanger method (F. Sanger, Science, 214, 1215, 1981) directly using the PCR product as a template to confirm that it was identical to the sequence in the database.

This PCR product was routinely transformed into the SUR2/YDC1 double gene disruption strain, and the transformants were screened on YPD plate containing 300 mg/L G418 (1% yeast extract, 2% polypeptone, 2% glucose, 2% agar) to give a SUR2/YDC1/SCS7 gene triple disruption strain.

### Example 6

### Preparation of Transformant Strains of the Yeast Ceramide Synthetic/Metabolic System Carrying a Human DES1-Expressing Plasmid

A strain expressing the DES1 gene (Example 1) or a strain expressing the DES1 gene and highly expressing the ISC1 gene (Example 2) was constructed using each gene disruption strain obtained in Examples 3-5 above.

I. SUR2 disruption (Example 3)+human DES1 gene expression (Example 1);

II. SUR2/SCS7 double disruption (Example 4)+human DES1 gene expression;

III. SUR2/SCS7/YDC1 triple disruption (Example 5)+human DES1 gene expression;

IV. SUR2/SCS7/YDC1 triple disruption (Example 5)+human DES1 gene expression and enhanced expression of ISC1 gene (Examples 1 and 2).

The human DES1-expressing plasmid of Example 1 and the ISC1-expressing plasmid of Example 2 were routinely transformed into the disruption strains obtained in Examples 3-5. Cells transformed with the DES1 gene and cells transformed with the DES1 and ISC1 genes were selected on complete minimal plates lacking uracil and complete minimal plates lacking uracil and tryptophan, respectively (SC-Ura and SC-Ura, Trp, respectively).

The expression of the human DES1 gene in the resulting transformant strains was verified by reverse transcription reaction of total RNA purified by an RNeasy kit (Qiagen) using Superscript II (Invitrogen) with primer Hdes1_R (SEQ ID NO: 31) and PCR (RT-PCR) of the reaction product using Ex-taq (Takara Bio) with Hdes1_F (SEQ ID NO: 32) and Hdes1_P (SEQ ID NO: 31).

SEQ ID NO: 31: 5′-TCCAGCACCATCTCTCCTTT-3′
SEQ ID NO: 32: 5′-AGTGGGTCTACACCGACCAG-3′.

The enhanced expression of the ISC1 gene was also verified by reverse transcription reaction using primer isc1R (SEQ ID NO: 14) and RT-PCR using primers isc1F (SEQ ID NO: 13) and isc1R (SEQ ID NO: 14).

### Example 7

### Analyses of Sphingoid Bases in the Transformant Strains of the Yeast Ceramide Synthetic/Metabolic System Carrying a Human DES1-Expressing Plasmid

The yeast SUR2 disruption strain expressing the human DES1 gene (I), the yeast SUR2/SCS7 double disruption strain expressing the human DES1 gene (II), and the yeast SUR2/SCS7/YDC1 triple disruption strain expressing the human DES1 gene (III) obtained in Example 6 were cultivated in liquid minimal medium containing tryptophan, histidine, leucine and lysine (SD+Trp, His, Leu, and Lys), and the yeast SUR2/SCS7/YDC1 triple disruption strain expressing the human DES1 and highly expressing ISC1 (IV) was cultivated in liquid minimal medium containing histidine, leucine and lysine (SD+His, Leu, and Lys) at 30° C. for 24 hours. Then, they were incubated under heat shock conditions at 37° C. for 90 minutes, and sphingoid bases were extracted from the cells and derivatized with dinitrophenol as described in a publication (Sperling et al., Journal of Biological chemistry, 273, 28590, 1998).

The sphingoid bases were analyzed by thin layer chromatography (TLC) and high-speed liquid chromatography (HPLC). The procedures are briefly described below.

Cells (wet weight 350 mg) were directly hydrolyzed in 3 ml of 1,4-dioxane/water, 1:1 (v/v) containing 10% (w/v) Ba(OH)₂ at 110° C. for 24 hours. Released sphingoid bases were extracted by separation into layers with chloroform/1,4-dioxane/water, 8:3:8 (v/v/v). The organic layers were washed with equal amounts of 0.1 M KOH and 0.5 M KCl, and then reacted with 0.2 ml of a 0.5% (v/v) solution of 1-fluoro-2,4-dinitrobenzene in methanol and 0.8 ml of 2M borate/KOH (pH 10.5) at 60° C. for 30 minutes to derivatize the sphingoid bases with dinitrophenol (DNP-derivatization). After the reaction, the resulting organic layers were dried in vacuo and the resulting DNP-derivatized sphingoid bases were dissolved in chloroform and then developed with chloroform/methanol, 9:1 (v/v) on silica gel 60 TLC plates. The DNP-derivatized sphingoid bases were observed as yellow spots (dark bleu under UV radiation).

Then, the DNP-derivatized sphingoid bases were recovered from the TLC plates and extracted with chloroform/methanol, 2:1 (v/v), and then separated into layers with chloroform/methanol/0.1 M KOH, 2:1:1 (v/v/v). The resulting organic layers were dried in vacuo, and then dissolved in methanol to prepare HPLC samples. HPLC was performed on a silica gel ODS column, eluting with a linear gradient of 80% methanol/acetonitrile/2-propanol (10:3:1, v/v/v) and 20% water to 0% water (flow rate 1 ml/min, 40 min), and UV absorption at 350 nm was monitored.

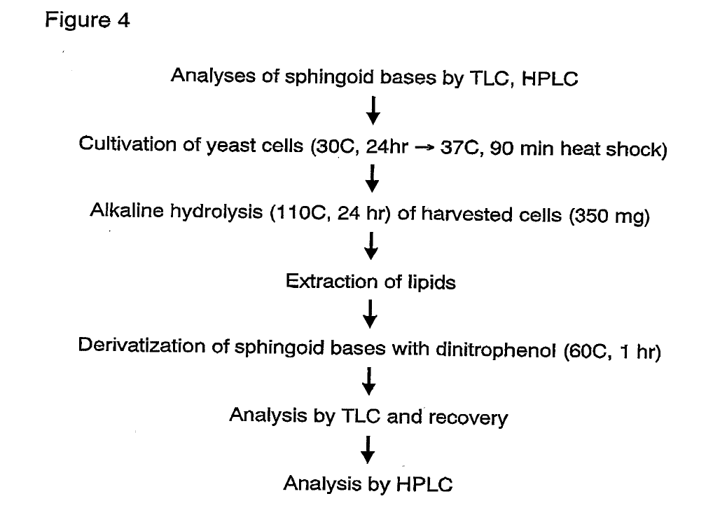

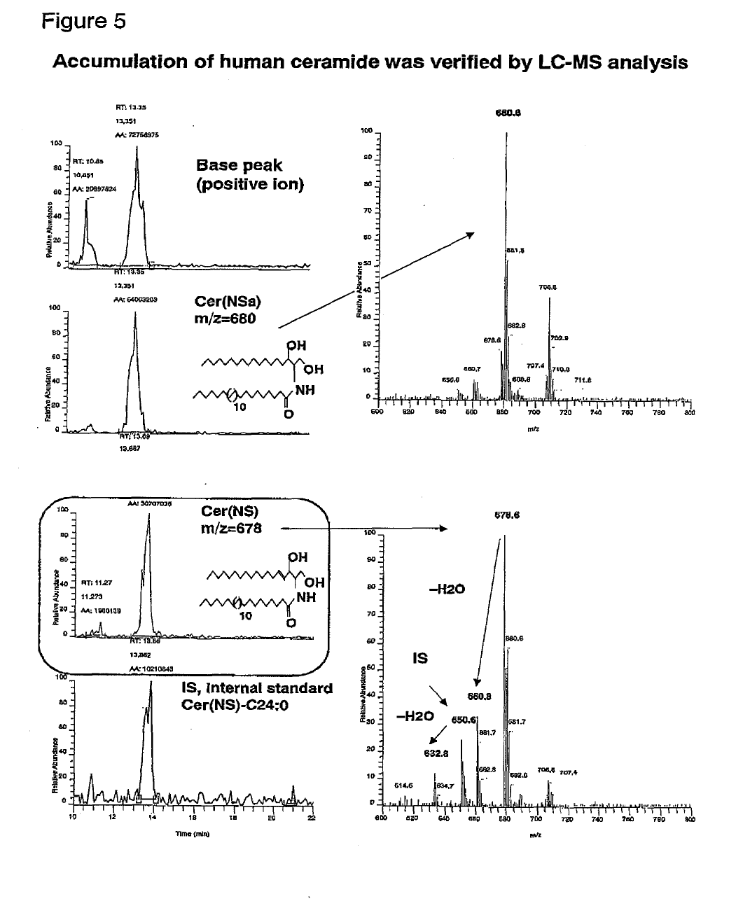

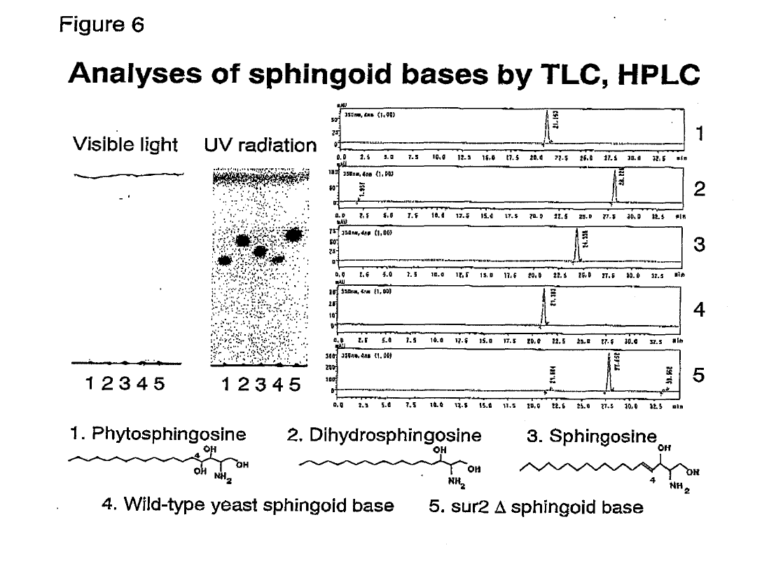

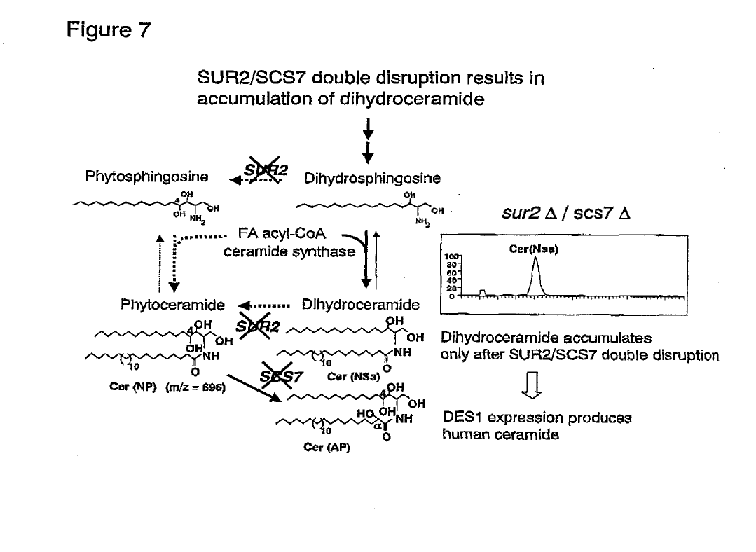

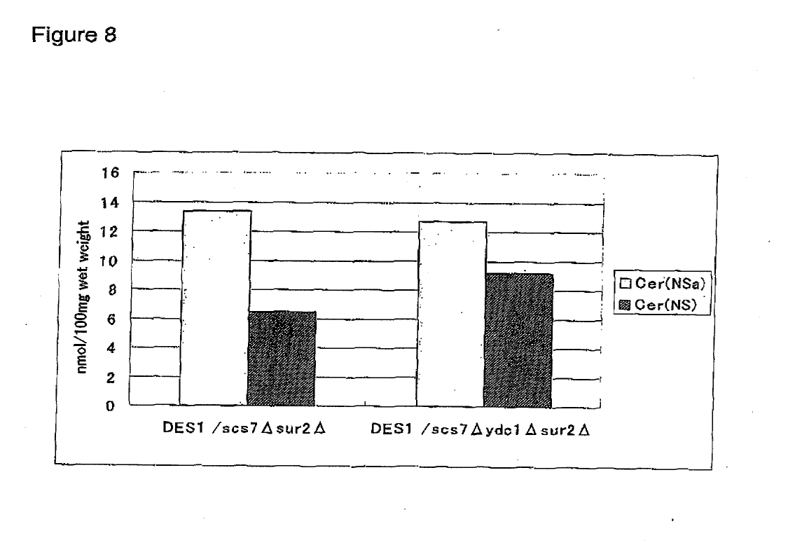

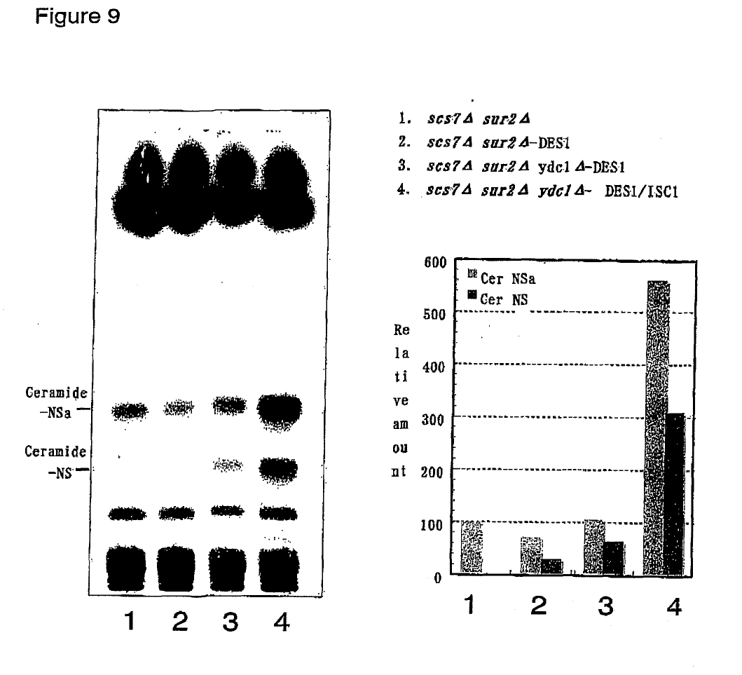

For easier understanding, a scheme for analyses of sphingoid bases by TLC and HPLC is shown in FIG. 4.

The amounts of sphingosine bases in cells were determined on the basis of the HPLC data obtained by similarly analyzing a predetermined amount of a synthetic sphingosine base purchased from Sigma. Table 1 shows the calculated amounts of sphingosine bases accumulated in 100 mg of cells. The amounts of sphingosine bases in cells increased in the order of the yeast SUR2 disruption strain expressing human DES1, the yeast SUR2/SCS7 double disruption strain expressing human DES1, the yeast SUR2/SCS7/YDC1 triple disruption strain expressing human DES1, and the yeast SUR2/SCS7/YDC1 triple disruption strain expressing human DES1 and highly expressing ISC1.

TABLE 1
Amounts of sphingosine bases in 100 mg of yeast cells

| | Amounts of sphingosine |
|---|---|
| | bases in μg |
| Yeast SUR2 disruption strain | 3.98 |
| expressing human DES1 | |
| Yeast SUR2/SCS7 double disruption | 6.08 |
| strain expressing human DES1 | |
| Yeast SUR2/SCS7/YDC1 triple | 8.35 |
| disruption strain expressing human | |
| DES1 | |
| Yeast SUR2/SCS7/YDC1 triple | 9.32 |
| disruption strain expressing human | |
| DES1 and highly expressing ISC1 | |

### Example 8

### Extraction of Yeast Lipids for LC-MS Analysis

The yeast transformants of ceramide synthetic pathway genes of Example 6 were cultivated in 250 ml of liquid minimal medium containing histidine, leucine, lysine, and tryptophan (SD+His, Leu, Lys and Trp, except that tryptophan was not contained for the strain highly expressing ISC1) at 30° C. with shaking at 140 rpm for 24 hours, and then incubated under heat shock conditions at 37° C. for 90 minutes. The cells were harvested and washed twice with sterilized water, and a suspension of 100 mg of the cells in 500 μl of sterilized water was prepared. The suspension was vigorously stirred with glass beads at room temperature for 5 minutes to disrupt the cells. Lipids were extracted by adding chloroform and methanol in a ratio of chloroform:methanol:suspension of 10:10:3. The extracts were centrifuged and the resulting supernatants were collected and concentrated/dried by blowing with nitrogen gas.

### Example 9

### Identification of the Molecular Species of Ceramides by LC-MS Analysis

The yeast lipids extracted in Example 8 were extracted with hexane/isopropanol (3:2 v/v), washed with 6.67% aqueous sodium sulfate, and then analyzed by the apparatus described in Japanese Patent Laid-open Publication No. 2003-28849 to identify the molecular species of ceramides. The results of the identification of the molecular species of ceramides in the yeast SUR2/SCS7 double disruption strain expressing the human DES1 gene are shown in FIG. 5.

As shown in FIG. 5, it was verified that ceramide NS (Cer(NS)) (Mw 678) had been synthesized in the yeast SUR2/SCS7 double disruption strain expressing the human DES1 gene.

It was also verified that ceramide NS (Cer(NS)) (Mw 678) had been synthesized in the yeast SUR2/SCS7/YDC1 triple disruption strain expressing human DES1, and the amounts of ceramide NS (Cer(NS)) accumulated in 100 mg of cells successively increased from 6.5 nmol in the yeast SUR2/SCS7 double disruption strain expressing the human DES1 gene to 9.2 mmol in the yeast SUR2/SCS7/YDC1 triple disruption strain expressing human DES1, as shown in FIG. 8.

### Example 10

### Analyses of Sphingoid Bases in the SUR2 Disruption Strain

The SUR2 disruption strain was analyzed for sphingoid bases by TLC and HPLC. The TLC and HPLC analyses were performed according to the method described in Example 8. The results are shown in FIG. 6.

The sphingoid base extracted from the SUR2 disruption strain (sample 5) was identified as dihydrosphingosine from the location of the HPLC peak agreeing with that of sample 2. In contrast, the sphingoid base extracted from wild-type yeast (sample 4) was identified as phytosphingosine from the location of the HPLC peak agreeing with that of sample 1. This shows that dihydrosphingosine is preferentially synthesized in the SUR2 disruption strain in contrast to wild-type yeast.

Sphingosine as a precursor of ceramide NS will be synthesized by introducing and expressing DES1 in this SUR2 disruption strain.

### Example 11

### Ceramide Analysis in the SUR2/SCS7 Disruption Strain

The SUR2/SCS7 double disruption strain was analyzed by LC-MS according to the method described in Example 8. The results are shown in FIG. 7. In the SUR2/SCS7 double disruption strain, accumulation of dihydroceramide (Cer(NSa)) was demonstrated, rather than a sphingoid base with an amide-linked fatty acid having a hydroxyl group at the α carbon (Cer ASa) or Cer(AP)). Human ceramide will be synthesized by expressing DES1 in this disruption strain.

### Example 12

### Ceramide Analysis Using Tritiated (³H) D-Erythro-Dihydrosphingosine

The yeast transformants of ceramide synthetic pathway genes described above were cultivated in liquid minimal medium containing histidine, leucine, lysine, and tryptophan (SD+His, Leu, Lys and Trp, except that tryptophan was not contained for the strain highly expressing ISC1) at 25° C. with shaking at 150 rpm for 24 hours, and then the yeast cells were harvested and suspended in liquid minimal medium to prepare 5 ml of a suspension (OD600=0.5). The suspension was incubated with 10 μl (10 μCi) of tritiated (3H) D-erythro-dihydrosphingosine overnight at 25° C. (Zanolari et al., The EMBO Journal, 19, 2824, 2000). The reaction was quenched with 200 μl of 250 mM NaF and 250 mM NaN₃, and then washed three times with ice-cooled sterilized water, and the cells were suspended in 66 μl of sterilized water.

The suspension was vigorously stirred with glass beads to disrupt the cells. Lipids were extracted by adding chloroform and methanol in a ratio of chloroform:methanol suspension of 10:10:3. The extracts were centrifuged and the resulting supernatants were collected and concentrated/dried by blowing with nitrogen gas. The samples were dissolved in 100 μl of chloroform-methanol-water (10:10:3), and reacted with 20 μl of a 0.6 N solution of NaOH in methanol at 300° C. for 90 min, and then neutralized with a 0.6 N acetic acid solution. The reaction solution was desalted by butanol extraction, and the resulting butanol layer (upper layer) was concentrated/dried by blowing with nitrogen gas.

The lipids were dissolved in 20 μl of chloroform-methanol (1:1), spotted on borate-impregnated thin layer chromatography (TLC) plates, and developed with chloroform-methanol (9:1) (Triola et al., Molecular Pharmacology, 66, 1671, 2004) After the development, radioactively labeled ceramides were analyzed by a Bioimage Analyzer (BAS). The results are shown in FIG. 9.

If dihydroceramide (CerNSa) in the SUR2/SCS7 double disruption strain is assumed to be 100%, ceramide NS (CerNS) increased in order from 30% in the yeast SUR2/SCS7 double disruption strain expressing the human DES1 gene was, 64% in the yeast SUR2/SCS7/YDC1 triple disruption strain expressing human DES1, and then 309% in the yeast SUR2/SCS7/YDC1 triple disruption strain expressing human DES1 and highly expressing ISC1.

1. A method for producing human ceramide in a yeast cell, comprising:
1) introducing the sphingoid Δ4-desaturase gene (DES1) by transformation of the yeast cell; and
2) abolishing the expression of the yeast sphinganine C4-hydroxylase gene (SUR2) by transformation of the yeast cell.

2. The method of claim 1 wherein the sphingoid Δ4-desaturase gene (DES1) has the amino acid sequence of SEQ ID NO: 2 or an amino acid sequence obtained by deletion, addition or substitution of one or more amino acid residues in SEQ ID NO: 2, and encodes a protein having sphingoid Δ4-desaturase activity.

3. The method of claim 1 wherein the yeast sphinganine C4-hydroxylase gene (SUR2) has the amino acid sequence of SEQ ID NO: 6 or amino acid sequence obtained by deletion, addition or substitution of one or more amino acid residues in SEQ ID NO: 6, and encodes a protein having sphinganine C4-hydroxylase activity.

4. The method of claim 1, further comprising abolishing the expression of the yeast sphingolipid α-hydroxylase gene (SCS7) by transformation of the yeast cell.

5. The method of claim 1 wherein the yeast sphingolipid α-hydroxylase gene has the amino acid sequence of SEQ ID NO: 8 or an amino acid sequence obtained by deletion, addition or substitution of one or more amino acid residues in SEQ ID NO: 8, and encodes a protein having sphingolipid α-hydroxylase activity.

6. The method of claim 1, further comprising abolishing the expression of the yeast alkaline dihydroceramidase gene (YDC1) by transformation of the yeast cell.

7. The method of claim 1 wherein the yeast alkaline dihydroceramidase gene (YDC1) has the amino acid sequence of SEQ ID NO: 10 or an amino acid sequence obtained by deletion, addition or substitution of one or more amino acid residues in SEQ ID NO: 10, and encodes a protein having alkaline dihydroceramidase activity.

8. The method of claim 1, further comprising enhancing the expression of the yeast inositol phosphosphigolipid phospholipase C gene (ISC1) by transformation of the yeast cell.

9. The method of claim 1 wherein the yeast inositol phosphosphingolipid phospholipase C gene (ISC1) has the amino acid sequence of SEQ ID NO: 4 or an amino acid sequence obtained by deletion, addition or substitution of one or more amino acid residues in SEQ ID NO: 4, and encodes a protein having inositol phosphosphingolipid phospholipase C activity.

10. A method for producing human ceramide in a yeast cell, comprising:
1) introducing the sphingoid Δ4-desaturase gene (DES1) by transformation of the yeast cell;
2) abolishing the expression of the yeast sphinganine C4-hydroxylase gene (SUR2) by transformation of the yeast cell;
3) abolishing the expression of the yeast sphingolipid α-hydroxylase gene (SCS7) by transformation of the yeast cell;
4) abolishing the expression of the yeast alkaline dihydroceramidase gene (YDC1) by transformation of the yeast cell; and
5) enhancing the expression of the yeast inositol phosphosphingolipid phospholipase C gene (ISC1) by transformation of the yeast cell.

11. The method of claim 10 wherein the yeast is selected from yeast species of the genus Saccharomyces.
